SEQUENCE LISTING (4 sequences)

SEQ ID NO: 1 - DNA, 18 bp, Artificial sequence
  Chemically snythesized forward primer for polymerase chain reaction amplication of Fas
aaaggctttgttcgaaag

SEQ ID NO: 2 - DNA, 20 bp, Artificial sequence
  Chemically snythesized reverse primer for polymerase chain reaction amplication of Fas
cactctagaccaagctttgg

SEQ ID NO: 3 - DNA, 19 bp, Artificial sequence
  Chemically synthesized forward primer for polymerase chain reaction amplification of 18S ribosomal subunit rRNA
cattcgtattgcgccgcta

SEQ ID NO: 4 - DNA, 19 bp, Artificial sequence
  Chemically synthesized reverse primer for polymerase chain reaction amplification of 18S ribosomal subunit rRNA
cgacggtatctgatcgtct

(19) United States
(12) Patent Application Publication
(10) Pub. No.: US 2007/0286867 A1
(43) Pub. Date: Dec. 13, 2007

(54) Combination Therapy

(75) Inventors: Patrick Gerard Johnston, Belfast (GB); Daniel Longley, Lisburn (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Fusion Antibodies Limited, Belfast (GB)

(21) Appl. No.: 10/580,662

(22) PCT Filed: Nov. 26, 2004
(86) PCT No.: PCT/GB04/05008

§ 371 Date: Oct. 10, 2006

(30) Foreign Application Priority Data
Nov. 26, 2003 (GB) 0327493.3

(51) Int. Cl. A61K 39/395; A61P 35/00

(52) U.S. Cl. 424/174.1

(57) ABSTRACT

The invention provides combination therapies and methods for the treatment of cancers associates with p53 mutations. The method comprises the separate, sequential or simultaneous administration of a therapeutically effective amount of a) a specific binding member, for example CH-11, which binds to a cell death receptor, for example Fas, or a nucleic acid encoding said binding member and b) a chemotherapeutic agent, wherein the chemotherapeutic agent is a topoisomerase inhibitor, for example CPT-1 or a thymidylate synthase inhibitor, for example TDX. Synergistic cytotoxic effects have been demonstrated using such combinations.

### FIELD OF THE INVENTION

This application relates to combination therapy and its use in methods of treatment. In particular, it relates to the treatment of cancer cells comprising a p53 mutation with a death receptor ligand, e.g. a FAS (CD95 or TNF receptor 2) receptor ligand, and a chemotherapeutic agent.

### BACKGROUND TO THE INVENTION

Breast, oesophageal, colorectal, all forms of GI cancer and head and neck cancers are highly malignant with overall 5-year survival rates of less than 50%. The clinical outcome of these patients is predetermined by the presence of widely disseminated tumour cells termed micrometastases with potential for metastatic growth, prior to clinical presentation. Approximately 50% of oesophageal cancer patients are selected for surgical therapy with 30% 5-year survival for this patient sub-group. Randomised clinical trials of neoadjuvant 5FU-based chemotherapy combined with fractionated radiotherapy have demonstrated improvements in survival of 10-20%, although the overall 5-year outcome for the treated groups remains at 30-35%. Those patients who demonstrate complete pathological response in their primary tumours as a result of neoadjuvant treatment have a five-year survival of 80%. Conversely, those patients who do not respond to 5FU-based chemotherapy are denied the opportunity for earlier treatment by surgery or a different neoadjuvant chemotherapeutic based regimen.

Colorectal cancer (CRC) is the second highest cause of cancer mortality in the western world. Approximately 40-50% of colon cancers will harbour mutations in the tumour suppressor gene p53. There is increasing evidence that not all p53 mutations result in absolute loss of function. Functional activities or properties of mutant proteins include retained wild-type activity [49], loss of function [50], gain of function [51, 52], dominant-negative effect [53] and temperature sensitivity. Two of the most prevalent p53 mutations in colon cancer occur at the codon ‘hotspots’ 175 and 248. These missense mutations result in the substitution of either histidine (R175H mutation) or tryptophan (R248W mutation) for arginine.

The most frequently used chemotherapeutic agents for the treatment of colorectal cancers are the fluoropyrimidine 5-fluorouracil (5-FU), the topoisomerase-I inhibitor Irinotecan (CPT-11) and the platinum agent Oxaliplatin. The thymidylate synthase inhibitor Tomudex (TDX) is also still used in the treatment of advanced colorectal cancer. 5-FU acts primarily by inhibiting the enzyme thymidylate synthase (TS) [40]. Because TS is a key enzyme in the de novo synthesis of thymidylate, its inhibition results in imbalances in intracellular dNTP pools and inhibition of DNA synthesis [41]. 5-FU also has direct effects on DNA and RNA, which contributes to its cytotoxicity [42]. CPT-11 is a prodrug that is hydrolysed to its active metabolite SN-38 by carboxylesterases [43]. It exerts its cytotoxic effect through the inhibition of topoisomerase-I [44]. Topo-I inhibitors stabilise the complex between topo-I and DNA which collide with moving DNA replication forks, leading to double stranded DNA breaks. Oxaliplatin is a third generation platinum cytotoxic in which a diaminocyclohexane (DACH) moiety replaces the amine groups present in cisplatin [44]. Although, like cisplatin, oxaliplatin also causes DNA-platinum adducts, it forms less of these than cisplatin and yet demonstrates more cytotoxicity. It is suggested that the oxaliplatin-DNA adducts are more lethal than cisplatin adducts [45]. Tomudex is a specific TS inhibitor. It is transported into cells via a reduced folate carrier and then undergoes extensive polyglutamation. The polyglutamated forms are up to 100 times more active then the parent compound [46]. 5-FU alone is used extensively as adjuvant chemotherapy in patients with early stage CRC [47]. Combinations of 5-FU together with either CPT-11 or Oxaliplatin are the standard of care for patients with advanced CRC [48].

Nevertheless, despite improvements in the efficacy of chemotherapy drugs used in the treatment of colorectal cancer, response rates are of the order of 45-50% for the most effective drug combinations.

The Fas/CD95 receptor is a 48 kDa member of the tumour necrosis factor receptor (TNFR) family [36]. The signalling members of the TNFR superfamily can be divided into two groups based on the composition of their cytoplasmic region. The death receptors (Fas/CD95 together with the receptors TNFR1, TNFR2, DR4 and DR5) contain a death domain in the cytoplasmic part of the receptor while the other group does not. This death domain is essential for transduction of the apoptotic signal. Binding of the Fas death receptor to its cognate ligand, called FasL, results in recruitment of FADD and caspase 8 to the receptor, and the formation of the death-inducing signalling complex (DISC) [17]. Active caspase 8 in turn activates downstream executioner caspases including caspase 3, which cleave a cassette of proteins resulting in cell death [37]. Caspase 8 also activates the mitochondrial cell death pathway through cleavage of the protein Bid. A variety of chemotherapeutic agents have been shown to cause up-regulation of the Fas/CD95 receptor in cancer cell lines. Fas/CD95 induction has also been documented following treatment of cancer cell lines with UV radiation [38]. The ability of chemotherapy drugs to induce the receptor has stimulated interest in targeting the Fas/CD95 death receptor with either therapeutic antibodies or peptides to enhance cell kill.

Thus, there is an urgent need for improved therapeutic strategies.

### SUMMARY OF THE INVENTION

As described herein, the present inventors have shown that by combining treatment using a death receptor ligand, such as an anti FAS antibody, with a thymidylate synthase inhibitor such as 5-FU, a topoisomerase inhibitor such as CPT-11, an antifolate drug, such as ralitrexed (RTX) or pemetrexed (MTA, Alimta), a platinum based cytotoxic such as oxaliplatin, a synergistic effect is achieved in the killing of cancer cells. However, the inventors have further shown that, for some chemotherapeutic agents, such as the platinum based cytotoxics, the synergistic cytotoxic effect is p53 dependent. As described in the Examples, the synergy observed for the combinations comprising such chemotherapeutic agents was not observed for corresponding p53 mutant cells. However, to the inventors' surprise, it was demonstrated that the synergistic cytoxic properties obtained using the combination of death receptor ligand with a chemotherapeutic agent was maintained for certain chemotherapeutic agents, such as RTX and CPT-11.

Accordingly, in a first aspect, the present invention provides a method of killing cancer cells having a p53 mutation, said method comprising the separate, sequential or simultaneous administration to said cells of a therapeutically effective amount of a) a specific binding member which binds to a cell death receptor or a nucleic acid encoding said binding member and (b) a chemotherapeutic agent, wherein the chemotherapeutic agent is a topoisomerase inhibitor or a thymidylate synthase inhibitor.

In a second aspect, the present invention provides a method of treating cancer cells having a p53 mutation comprising the separate, sequential or simultaneous administration to a mammal in need thereof of a therapeutically effective amount of a) a specific binding member which binds to a cell death receptor or a nucleic acid encoding said binding member and (b) a chemotherapeutic agent, wherein the chemotherapeutic agent is a topoisomerase inhibitor or a thymidylate synthase inhibitor.

The specific binding member and the chemotherapeutic agent may be administered simultaneously, sequentially or simultaneously. In preferred embodiments of the invention, the chemotherapeutic agent is administered prior to the specific binding member.

In a third aspect, there is provided the use of

(a) a specific binding member which binds to a cell death receptor or a nucleic acid encoding said binding member and

(b) a chemotherapeutic agent, wherein the chemotherapeutic agent is a topoisomerase inhibitor or a thymidylate synthase inhibitor in the preparation of a medicament for treating cancer, wherein the cancer cells comprise a p53 mutation.

In a fourth aspect, there is provided a product comprising a) a specific binding member which binds to a cell death receptor or a nucleic acid encoding said binding member and (b) a chemotherapeutic agent as a combined preparation for the simultaneous, separate or sequential use in the treatment of cancer, wherein the chemotherapeutic agent is a topoisomerase inhibitor or a thymidylate synthase inhibitor, and wherein the cancer cells comprise a p53 mutation.

According to a fifth aspect, there is provided a pharmaceutical composition for the treatment of a cancer characterised by the presence of a p53 mutation, wherein the composition comprises a) a specific binding member which binds to a cell death receptor or a nucleic acid encoding said binding member and (b) a chemotherapeutic agent, wherein the chemotherapeutic agent is a topoisomerase inhibitor or a thymidylate synthase inhibitor and (c) a pharmaceutically acceptable excipient, diluent or carrier.

In a sixth aspect, there is provided a kit for the treatment of a cancer characterised by the presence of a p53 mutation, said kit comprising a) a specific binding member which binds to a cell death receptor or a nucleic acid encoding said binding member and (b) a chemotherapeutic agent, wherein the chemotherapeutic agent is a topoisomerase inhibitor or a thymidylate synthase inhibitor and (c) instructions for the administration of (a) and (b) separately, sequentially or simultaneously.

Preferred thymidylate synthase inhibitors for use in the invention are antifolate thymidylate synthase inhibitors, such as ralitrexed (TDX) or pemetrexed (MTA). Preferred topoisomerase inhibitors for use in the invention are topoisomerase I inhibitors, such as camptothecins, such as CPT-11.

In a preferred embodiment of the invention, the chemotherapeutic agent is an antifolate, such as ralitrexed (TDX) or pemetrexed (MTA) or a topoisomerase-I inhibitor, such as CPT-11 or Particularly preferred examples of antifolates and topoisomerase-I inhibitors for use in the invention are TDX and irinotecan (CPT-11). Unless, the context demand otherwise, reference to CPT-11 should be taken to encompass CPT-11 or its active metabolite SN-38.

The invention may be used to treat any cancer comprising cells having a p53 mutation. The mutation may partially or totally inactivate p53 in a cell. In one embodiment of the invention, the p53 mutation is a p53 mutation, which totally inactivates p53. In another embodiment, the p53 mutation is a missense mutation resulting in the substitution of histidine (R175H mutation). In another embodiment, the p53 mutation is a missense mutation resulting in the substitution of tryptophan (R248W mutation) for arginine.

In preferred embodiments of the invention, the cancer is one or more of colorectal, breast, ovarian, cervical, gastric, lung, liver, skin and myeloid (e.g. bone marrow) cancer. In a particular embodiment of the invention, the cancer is a colorectal cancer.

The binding member for use in the invention may bind to any death receptor. Death receptors include, Fas, TNFR, DR-3, DR-4 and DR-5. In preferred embodiments of the invention, the death receptor is FAS.

In preferred embodiments of the invention, the binding member is an antibody or a fragment thereof.

In particularly preferred embodiments, the binding member is the FAS antibody CH11 (Yonehara, S., Ishii, A. and Yonehara, M. (1989) J. Exp. Med. 169, 1747-1756) (available commercially e.g. from Upstate Biotechnology, Lake Placid, N.Y.).

In preferred embodiments, the binding member comprises at least one human constant region.

The concentrations of binding members and chemotherapeutic agents used are preferably sufficient to provide a synergistic effect. Synergism is preferably defined as an RI of greater than unity using the method of Kern as modified by Romaneli (13, 14). The RI may be calculated as the ratio of expected cell survival (Sₑₓₚ, defined as the product of the survival observed with drug A alone and the survival observed with drug B alone) to the observed cell survival (S_{obs}) for the combination of A and B (RI=Sₑₓₚ/S_{obs}). Synergism may then be defined as an RI of greater than unity.

In preferred embodiments of the invention, said specific binding member and chemotherapeutic agent are provided in concentrations sufficient to produce an RI of greater than 1.5, more preferably greater than 2.0, most preferably greater than 2.25.

The combined medicament thus preferably produces a synergistic effect when used to treat tumour cells having a p53 mutant genotype.

A seventh aspect of the present invention therefore provides a medicament for use in treating p53 mutant tumour cells, the medicament comprising at least one antibody directed at FAS receptor and at least one cancer chemotherapeutic agent, wherein the chemotherapeutic agent is a topoisomerase inhibitor or a thymidylate synthase inhibitor.

Preferred features of each aspect of the invention are as for each of the other aspects mutatis mutandis.

### DETAILED DESCRIPTION

### Binding Members

In the context of the present invention, a “binding member” is a molecule which has binding specificity for another molecule, in particular a receptor, in particular a death receptor. A binding member of the invention and for use in the invention may be any moiety, for example an antibody or ligand, which can bind to a death receptor.

### Antibodies

An “antibody” is an immunoglobulin, whether natural or partly or wholly synthetically produced. The term also covers any polypeptide, protein or peptide having a binding domain which is, or is homologous to, an antibody binding domain. These can be derived from natural sources, or they may be partly or wholly synthetically produced. Examples of antibodies are the immunoglobulin isotypes and their isotypic subclasses and fragments which comprise an antigen binding domain such as Fab, scFv, Fv, dAb, Fd; and diabodies.

The binding member of the invention may be an antibody such as a monoclonal or polyclonal antibody, or a fragment thereof. The constant region of the antibody may be of any class including, but not limited to, human classes IgG, IgA, IgM, IgD and IgE. The antibody may belong to any sub class e.g. IgG1, IgG2, IgG3 and IgG4. IgG1 is preferred.

As antibodies can be modified in a number of ways, the term “antibody” should be construed as covering any binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

It has been shown that fragments of a whole antibody can perform the function of binding antigens. Examples of such binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341:544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., Science 242:423-426 (1988); Huston et al., PNAS USA 85:5879-5883 (1988)); (viii) bispecific single chain Fv dimers (PCT/US92/09965) and (ix) “diabodies”, multivalent or multispecific fragments constructed by gene fusion (WO94/13804; P. Hollinger et al., Proc. Natl. Acad. Sci. USA 90:6444-6448 (1993)).

A fragment of an antibody or of a polypeptide for use in the present invention generally means a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

A “derivative” of such an antibody or polypeptide, or of a fragment antibody means an antibody or polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, preferably while providing a peptide having death receptor, e.g. FAS neutralisation and/or binding activity. Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

The term “antibody” includes antibodies which have been “humanised”. Methods for making humanised antibodies are known in the art. Methods are described, for example, in Winter, U.S. Pat. No. 5,225,539. A humanised antibody may be a modified antibody having the hypervariable region of a monoclonal antibody and the constant region of a human antibody. Thus the binding member may comprise a human constant region.

The variable region other than the hypervariable region may also be derived from the variable region of a human antibody and/or may also be derived from a monoclonal antibody. In such case, the entire variable region may be derived from murine monoclonal antibody and the antibody is said to be chimerised. Methods for making chimerised antibodies are known in the art. Such methods include, for example, those described in U.S. patents by Boss (Celltech) and by Cabilly (Genentech). See U.S. Pat. Nos. 4,816,397 and 4,816,567, respectively.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

A typical antibody for use in the present invention is a humanised equivalent of CH11 or any chimerised equivalent of an antibody that can bind to the FAS receptor and any alternative antibodies directed at the FAS receptor that have been chimerised and can be use in the treatment of humans. Furthermore, the typical antibody is any antibody that can cross-react with the extracellular portion of the FAS receptor and either bind with high affinity to the FAS receptor, be internalised with the FAS receptor or trigger signalling through the FAS receptor.

### Production of Binding Members

The binding members for use in the present invention may be generated wholly or partly by chemical synthesis. The binding members can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Another convenient way of producing a binding member suitable for use in the present invention is to express nucleic acid encoding it, by use of nucleic acid in an expression system. Thus the present invention further provides the use of (a) nucleic acid encoding a specific binding member which binds to a cell death receptor and (b) a chemotherapeutic agent in the preparation of a medicament for treating cancer.

Nucleic acid for use in accordance with the present invention may comprise DNA or RNA and may be wholly or partially synthetic. In a preferred aspect, nucleic acid for use in the invention codes for a binding member of the invention as defined above. The skilled person will be able to determine substitutions, deletions and/or additions to such nucleic acids which will still provide a binding member suitable for use in the present invention.

Nucleic acid sequences encoding a binding member for use with the present invention can be readily prepared by the skilled person using the information and references contained herein and techniques known in the art (for example, see Sambrook, Fritsch and Maniatis, “Molecular Cloning”, A Laboratory Manual, Cold Spring Harbor Laboratory Press, 1989, and Ausubel et al, Short Protocols in Molecular Biology, John Wiley and Sons, 1992), given the nucleic acid sequences and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding antibody fragments may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the sequences can be made, e.g. using site directed mutagenesis, to lead to the expression of modified peptide or to take account of codon preferences in the host cells used to express the nucleic acid.

The nucleic acid may be comprised as construct(s) in the form of a plasmid, vector, transcription or expression cassette which comprises at least one nucleic acid as described above. The construct may be comprised within a recombinant host cell which comprises one or more constructs as above. Expression may conveniently be achieved by culturing under appropriate conditions recombinant host cells containing the nucleic acid. Following production by expression a specific binding member may be isolated and/or purified using any suitable technique, then used as appropriate.

Binding members-encoding nucleic acid molecules and vectors for use in accordance with the present invention may be provided isolated and/or purified, e.g. from their natural environment, in substantially pure or homogeneous form, or, in the case of nucleic acid, free or substantially free of nucleic acid or genes origin other than the sequence encoding a polypeptide with the required function.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of antibodies and antibody fragments in prokaryotic cells such as E. coli is well established in the art. For a review, see for example Pluckthun, Bio/Technology 9:545-551 (1991). Expression in eukaryotic cells in culture is also available to those skilled in the art as an option for production of a binding member, see for recent review, for example Reff, Curr. Opinion Biotech. 4:573-576 (1993); Trill et al., Curr. Opinion Biotech. 6:553-560 (1995).

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Sambrook et al., Molecular Cloning: A Laboratory Manual: 2nd Edition, Cold Spring Harbor Laboratory Press (1989). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. eds., Short Protocols in Molecular Biology, 2nd Edition, John Wiley & Sons (1992).

The nucleic acid may be introduced into a host cell by any suitable means. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells under conditions for expression of the gene.

The nucleic acid may be integrated into the genome (e.g. chromosome) of the host cell. Integration may be promoted by inclusion of sequences which promote recombination with the genome in accordance with standard techniques. The nucleic acid may be on an extra-chromosomal vector within the cell, or otherwise identifiably heterologous or foreign to the cell.

### Chemotherapeutic Agents

As described above, the present invention is based on the surprising demonstration that, contrary to the synergism demonstrated for antineoplastic combination therapies such as CH-11 and cisplatin, which is p53 dependent, the synergistic cytotoxic effects of combination therapies comprising a death receptor ligand and a topoisomerase inhibitor or a thymidylate synthase inhibitor is p53 independent.

Accordingly, the invention provides novel effective drug combinations for the treatment of p53 mutant tumours.

Any suitable a topoisomerase inhibitor or thymidylate synthase inhibitor may be used in the invention.

Examples of thymidylate synthase inhibitor antifolates include fluoropyrimidines such as 5-FU and antifolates such as RTX(TDX) and MTA. Examples of topoisomerase inhibitors include topoisomerase-I inhibitors, such as camptothecins and topoisomerase-II inhibitors.

Preferred topoisomerase inhibitors or thymidylate synthase inhibitors for use in the invention are those agents which demonstrate synergistic cytotoxic properties in combination with death receptor ligands such as CH-11 on p53 mutant cells, for example p53 null cells, preferably with an RI of greater than 1.5, preferably greater than 2.0.

In one particularly preferred embodiment, the agent is CPT-11.

In another particularly preferred embodiment, the agent is TDX.

### Treatment

Treatment” includes any regime that can benefit a human or non-human animal. The treatment may be in respect of an existing condition or may be prophylactic (preventative treatment). Treatment may include curative, alleviation or prophylactic effects.

“Treatment of cancer” includes treatment of conditions caused by cancerous growth and includes the treatment of neoplastic growths or tumours. Examples of tumours that can be treated using the invention are, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, cervical and ovarian carcinoma, lymphomas, including Hodgkin and non-Hodgkin lymphomas, neuroblastoma, melanoma, myeloma, Wilms tumor, and leukemias, including acute lymphoblastic leukaemia and acute myeloblastic leukaemia, gliomas and retinoblastomas.

The compositions and methods of the invention may be particularly useful in the treatment of existing cancer and in the prevention of the recurrence of cancer after initial treatment or surgery.

### Administration

Binding members and chemotherapeutic agents may be administered simultaneously, separately or sequentially.

Where administered separately or sequentially, they may be administered within any suitable time period e.g. within 1, 2, 3, 6, 12, 24, 48 or 72 hours of each other. In preferred embodiments, they are administered within 6, preferably within 2, more preferably within 1, most preferably within 20 minutes of each other.

In a preferred embodiment, they are administered as a pharmaceutical composition, which will generally comprise a suitable pharmaceutical excipient, diluent or carrier selected dependent on the intended route of administration.

Binding members and chemotherapeutic agents of and for use in the present invention may be administered to a patient in need of treatment via any suitable route. The precise dose will depend upon a number of factors, including the precise nature of the member (e.g. whole antibody, fragment or diabody) and chemotherapeutic agent.

Some suitable routes of administration include (but are not limited to) oral, rectal, nasal, topical (including buccal and sublingual), vaginal or parenteral (including subcutaneous, intramuscular, intravenous, intradermal, intrathecal and epidural) administration. Intravenous administration is preferred.

It is envisaged that injections (intravenous) will be the primary route for therapeutic administration of compositions although delivery through a catheter or other surgical tubing is also envisaged. Liquid formulations may be utilised after reconstitution from powder formulations.

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The binding member, agent, product or composition may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al, Biopolymers 22(1): 547-556, 1985), poly (2-hydroxyethyl-methacrylate) or ethylene vinyl acetate (Langer et al, J. Biomed. Mater. Res. 15: 167-277, 1981, and Langer, Chem. Tech. 12:98-105, 1982). Liposomes containing the polypeptides are prepared by well-known methods: DE 3,218, 121A; Epstein et al, PNAS USA, 82: 3688-3692, 1985; Hwang et al, PNAS USA, 77: 4030-4034, 1980; EP-A-0052522; E-A-0036676; EP-A-0088046; EP-A-0143949; EP-A-0142541; JP-A-83-11808; U.S. Pat. Nos. 4,485,045 and 4,544,545. Ordinarily, the liposomes are of the small (about 200-800 Angstroms) unilamellar type in which the lipid content is greater than about 30 mol. % cholesterol, the selected proportion being adjusted for the optimal rate of the polypeptide leakage.

Examples of the techniques and protocols mentioned above and other techniques and protocols which may be used in accordance with the invention can be found in Remington's Pharmaceutical Sciences, 16th edition, Oslo, A. (ed), 1980.

The binding member, agent, product or composition may be administered in a localised manner to a tumour site or other desired site or may be delivered in a manner in which it targets tumour or other cells. Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

### Pharmaceutical Compositions

As described above, the present invention extends to pharmaceutical composition for the treatment of a cancer characterised by the presence of a p53 mutation, wherein the composition comprises a) a specific binding member which binds to a cell death receptor or a nucleic acid encoding said binding member and (b) a chemotherapeutic agent, wherein the chemotherapeutic agent is a thymidylate synthase inhibitor, a topoisomerase-I inhibitor or a fluoropyrimidine. Pharmaceutical compositions according to the present invention, and for use in accordance with the present invention may comprise, in addition to active ingredients, a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

The formulation may be a liquid, for example, a physiologic salt solution containing non-phosphate buffer at pH 6.8-7.6, or a lyophilised powder.

### Dose

The binding members, agents, products or compositions are preferably administered to an individual in a “therapeutically effective amount”, this being sufficient to show benefit to the individual. The actual amount administered, and rate and time-course of administration, will depend on the nature and severity of what is being treated.

As described herein, the concentrations are preferably sufficient to show a synergistic effect. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

The optimal dose can be determined by physicians based on a number of parameters including, for example, age, sex, weight, severity of the condition being treated, the active ingredient being administered and the route of administration. For example, with respect to binding members, in general, a serum concentration of polypeptides and antibodies that permits saturation of receptors is desirable. A concentration in excess of approximately 0.1 nM is normally sufficient. For example, a dose of 100 mg/m² of antibody provides a serum concentration of approximately 20 nM for approximately eight days.

As a rough guideline, doses of antibodies may be given in amounts of 1 ng/kg-500 mg/kg of patient weight. Equivalent doses of antibody fragments should be used at the same or more frequent intervals in order to maintain a serum level in excess of the concentration that permits saturation of death receptor.

Doses of the binding members may be given at any suitable dose interval e.g. daily, once, twice or thrice weekly.

For example, the periods of administration of a humanised antibody could be from 1 bolus injection to weekly administration for up to one year in combination with chemotherapeutic agents. The likely dose is upwards of 1 mg/per kg/per patient.

Doses of chemotherapeutic agent will depend on the factors described above but preferably are administered in doses which are within the normal range or, preferably, at a lower concentration than the normal range, wherein the normal range is the range of concentrations at which the chemotherapeutic agent is usually administered in the absence of other therapeutic agents.

It is anticipated that in embodiments of the invention the binding members and chemotherapeutic agent could be given in combination with other forms of chemotherapy or indeed radiotherapy.

Thus, in a further aspect of the invention, there is provided a method of killing p 53 mutant cancer cells comprising administration of a therapeutically effective amount of a) a specific binding member which binds to a cell death receptor or a nucleic acid encoding said binding member, (b) a chemotherapeutic agent, wherein the chemotherapeutic agent is an antifolate, a thymidylate synthase inhibitor, or a a topoisomerase-I inhibitor (c) radoiotherapy treatment.

In a eleventh aspect, the present invention provides a method of treating cancer characterised by the presence of p53 mutant cells, said method comprising administration of a therapeutically effective amount of a) a specific binding member which binds to a cell death receptor or a nucleic acid encoding said binding member, (b) a chemotherapeutic agent, wherein the chemotherapeutic agent is a an antifolate, a thymidylate synthase inhibitor, or a topoisomerase-I inhibitor and (c) radoiotherapy treatment. to a mammal in need thereof.

The specific binding member and the radiotherapy may be administered simultaneously, sequentially or simultaneously. In preferred embodiments of the invention, the chemotherapeutic agent is administered prior to the radiotherapy.

The invention will now be described further in the following non-limiting examples. Reference is made to the accompanying drawings in which:

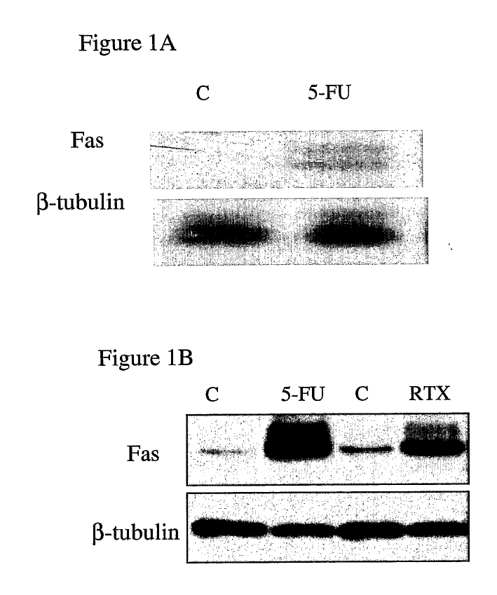

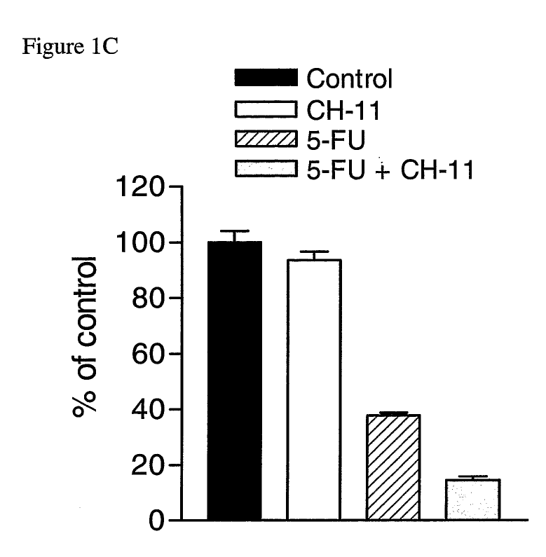

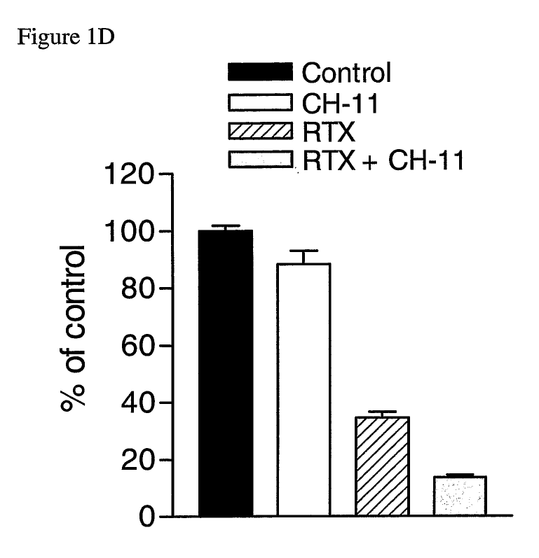

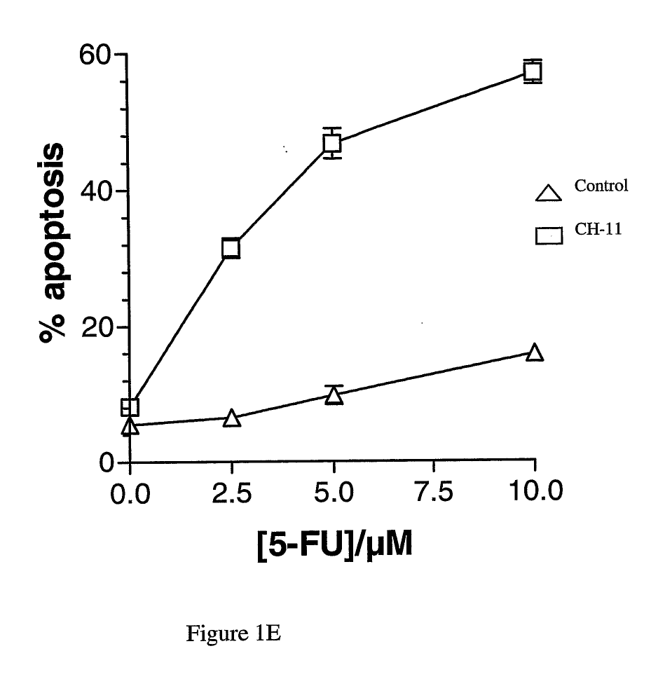

FIG. 1A illustrates Northern blot analysis of Fas mRNA expression in MCF-7 cells 48 hours after treatment with no drug (C) or 5 μM 5-FU. Equal loading was assessed by analysing β-tubulin mRNA expression.

FIG. 1B illustrates Western blot analysis of Fas expression in MCF-7 cells 72 hours after treatment with no drug (C), 51M 5-FU or 25 nM RTX. Equal loading was assessed by analysing P-tubulin expression.

FIG. 1C illustrates MTT cell viability assays in MCF-7 cells treated with no drug (control), CH-11 alone (250 ng/ml), 5-FU alone (5 μM), or co-treated with 5-FU and CH-11. The decrease in cell viability for the combined treatment was highly synergistic (RI=2.40, p<0.0005).

FIG. 1D illustrates MTT cell viability assays in MCF-7 cells treated with no drug (control), CH-11 alone (250 ng/ml), RTX alone (25 nM), or co-treated with RTX and CH-11. The decrease in cell viability for the combined treatment was highly synergistic (RI=2.22, p<0.0005).

FIG. 1E illustrates analysis of apoptosis in 5-FU and CH-11 co-treated MCF-7 cells.

FIG. 1F illustrates analysis of apoptosis in RTX and CH-11 co-treated MCF-7 cells. Apoptosis was assessed by analysing the sub-G₁/G₀ fraction of propidium iodide stained cells by flow cytometry. For both the MTT and flow cytometric analyses the cells were pre-treated with each chemotherapeutic drug for 72 hours followed by CH-11 for a further 24 hours.

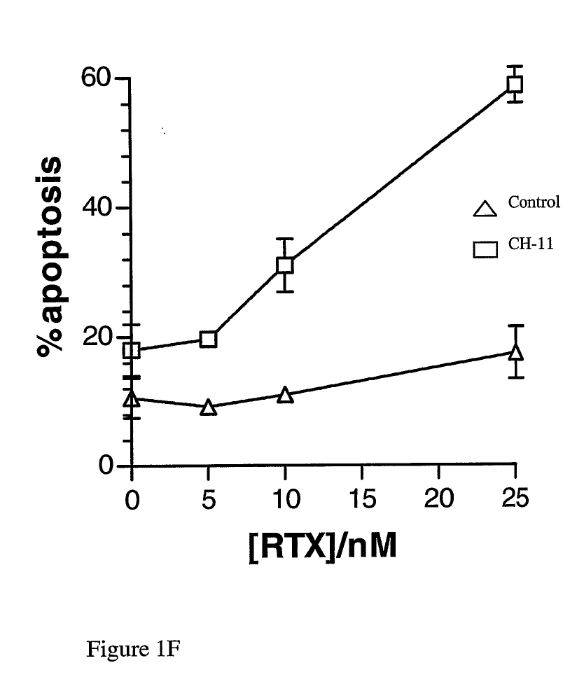

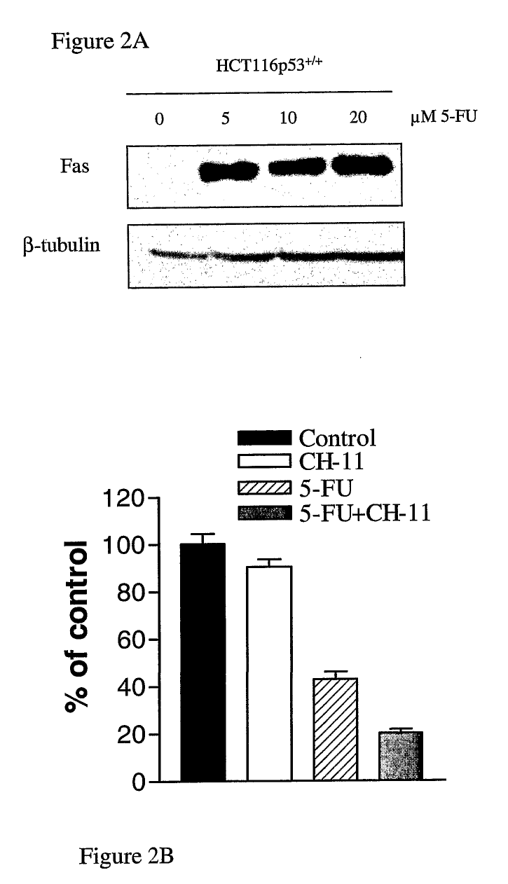

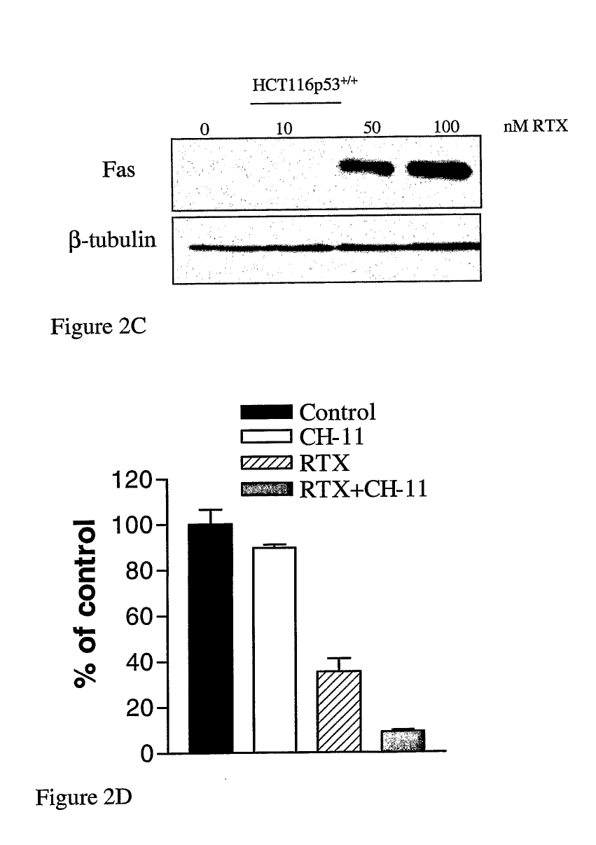

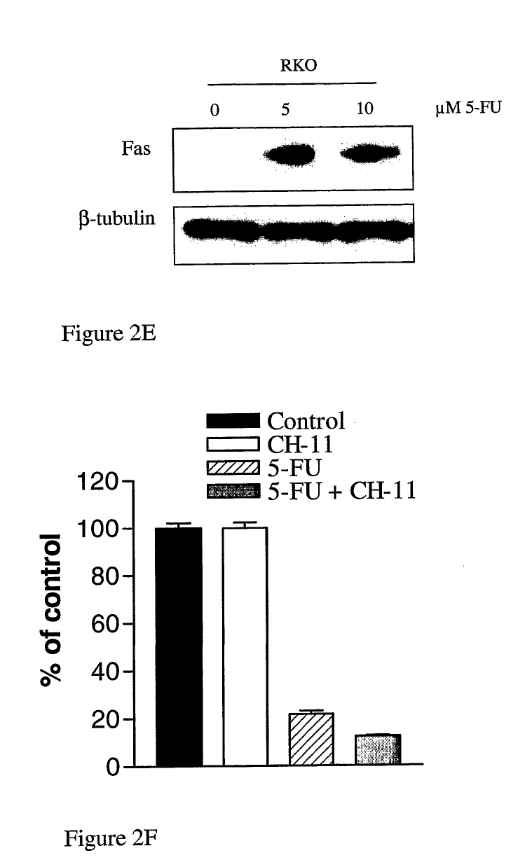

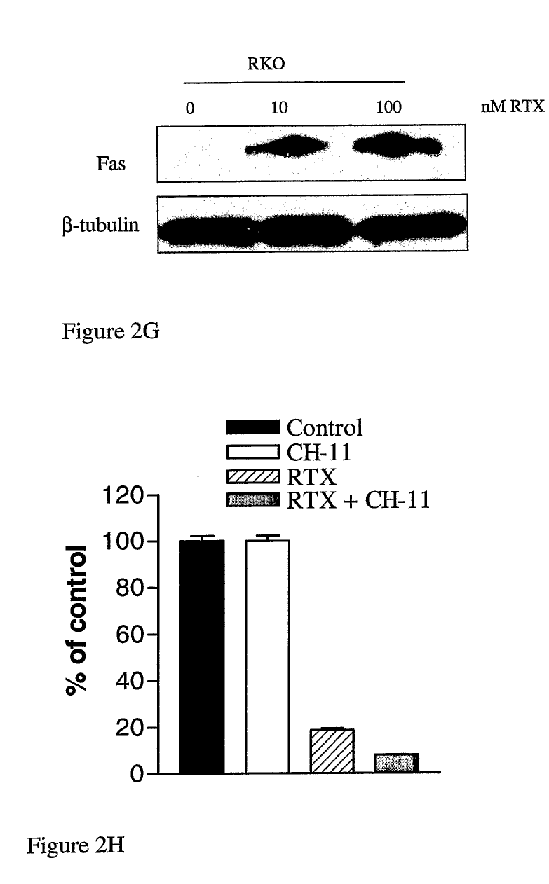

FIG. 2A illustrates Western blot analysis of Fas expression in HCT116p53^{+/+} cells treated with a range of concentrations of 5-FU for 48 hours.

FIG. 2B illustrates MTT cell viability assays in HCT116p53^{+/+} cells treated with no drug (control), CH-11 alone (250 ng/ml), 5-FU alone (5 μM), or co-treated with 5-FU and CH-11. The decrease in cell viability for the combined treatment was synergistic (RI=1.92, p<0.005).

FIG. 2C illustrates Western blot analysis of Fas expression in HCT116p53^{+/+} cells treated with a range of concentrations of RTX for 48 hours.

FIG. 2D illustrates MTT cell viability assays in HCT116p53^{+/+} cells treated with no drug (control), CH-11 alone (250 ng/ml), RTX alone (50 nM), or co-treated with RTX and CH-11. The decrease in cell viability for the combined treatment was highly synergistic (RI=3.44, p<0.0005).

FIG. 2E illustrates Western blot analysis of Fas expression in RKO cells treated with a range of concentrations of 5-FU for 48 hours.

FIG. 2F illustrates MTT cell viability assays in RKO cells treated with no drug (control), CH-11 alone (250 ng/ml), 5-FU alone (5 μM), or co-treated with 5-FU and CH-11. The decrease in cell viability for the combined treatment was synergistic (RI=1.74, p<0.005).

FIG. 2G illustrates Western blot analysis of Fas expression in RKO cells treated with a range of concentrations of RTX for 48 hours.

FIG. 2H illustrates MTT cell viability assays in RKO cells treated with no drug (control), CH-11 alone (250 ng/ml), RTX alone (5 nM), or co-treated with RTX and CH-11. The decrease in cell viability for the combined treatment was highly synergistic (RI=2.31, p<0.0005). Equal loading of Western blots was assessed by analysing β-tubulin expression. For each combined treatment the cells were pre-treated with chemotherapeutic drug for 72 hours followed by CH-11 for a further 24 hours.

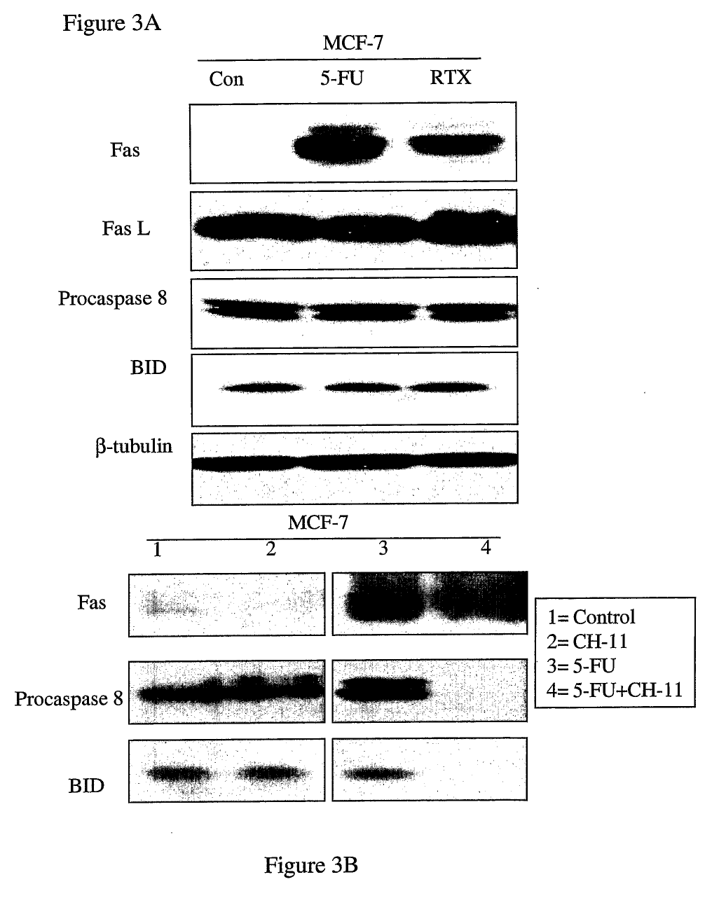

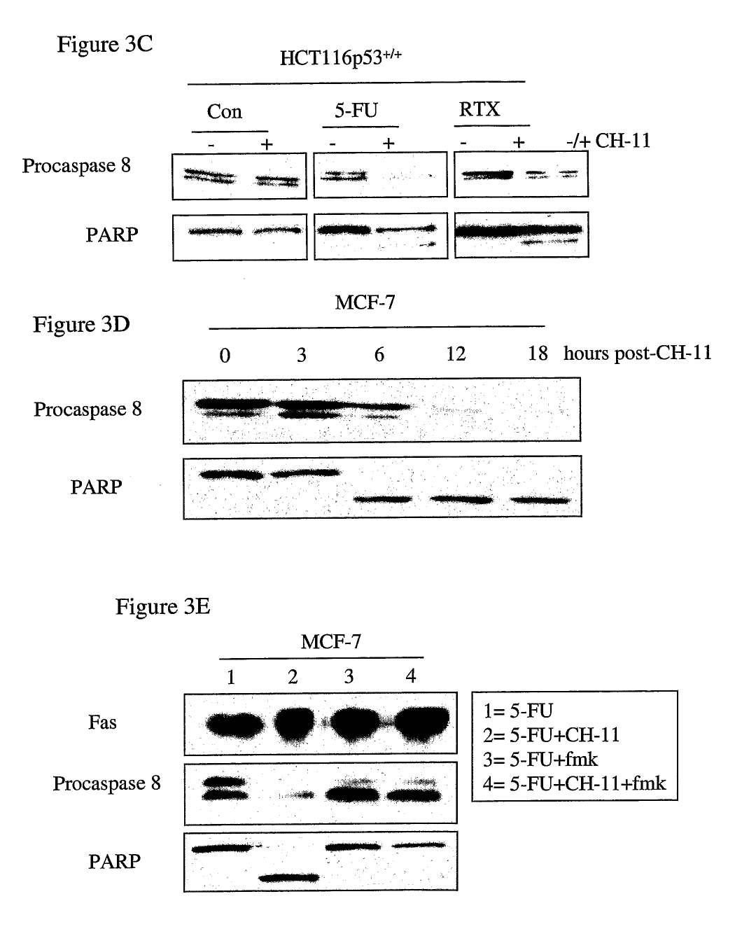

FIG. 3A illustrates Western blot analysis of Fas, FasL, procaspase 8 and BID expression in MCF-7 cells treated with IC₆₀ doses of 5-FU (5 μM) and RTX (25 nM) for 72 hours. Equal loading was assessed using a β-tubulin antibody.

FIG. 3B illustrates Western blot analysis of Fas, procaspase 8 and BID expression in MCF-7 cells treated no drug (control), CH-11 alone (250 ng/ml), 5-FU alone (5 μM) for 96 hours, or co-treated with 5-FU for 72 hours followed by CH-11 for a further 24 hours. Co-treatment with 5-FU and CH-11 resulted in activation of caspase 8 and BID as indicated by processing of procaspase 8 and full-length BID (lane 4).

FIG. 3C illustrates Western blot analysis of procaspase 8 and PARP expression in HCT116p53^{+/+} cells treated with no drug (control), 5 μM 5-FU or 50 nM RTX alone or in combination with 250 ng/ml CH-11.

FIG. 3D illustrates Western blot analysis examining the kinetics of caspase 8 activation and PARP cleavage in MCF-7 cells treated for 72 hours with 5 μM 5-FU followed by 250 ng/ml CH-11 for the indicated times.

FIG. 3E illustrates Western blot analysing Fas, procaspase 8 and PARP expression in MCF-7 cells treated with 51M 5-FU for 72 hours followed by 250 ng/ml CH-11, 10 μM IETD-fmk, or a combination of CH-11 and IETD-fmk for 24 hours.

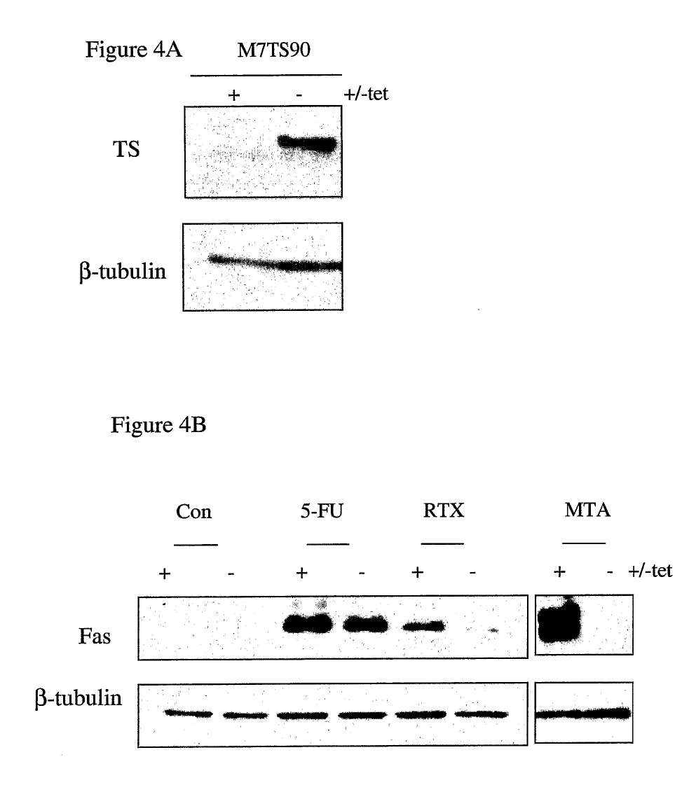

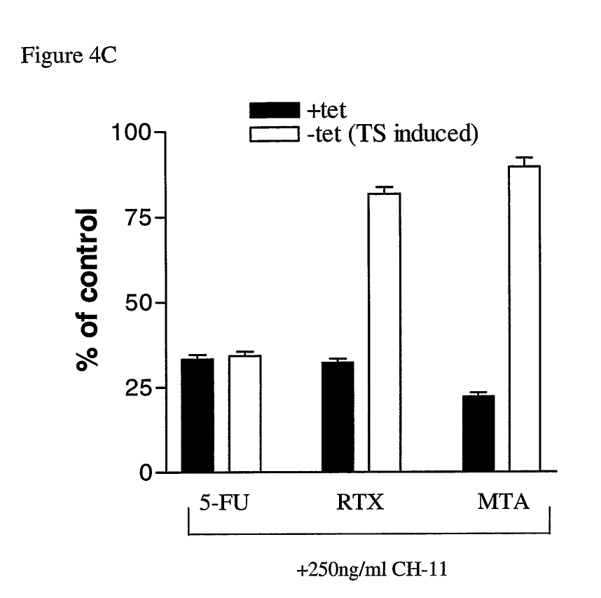

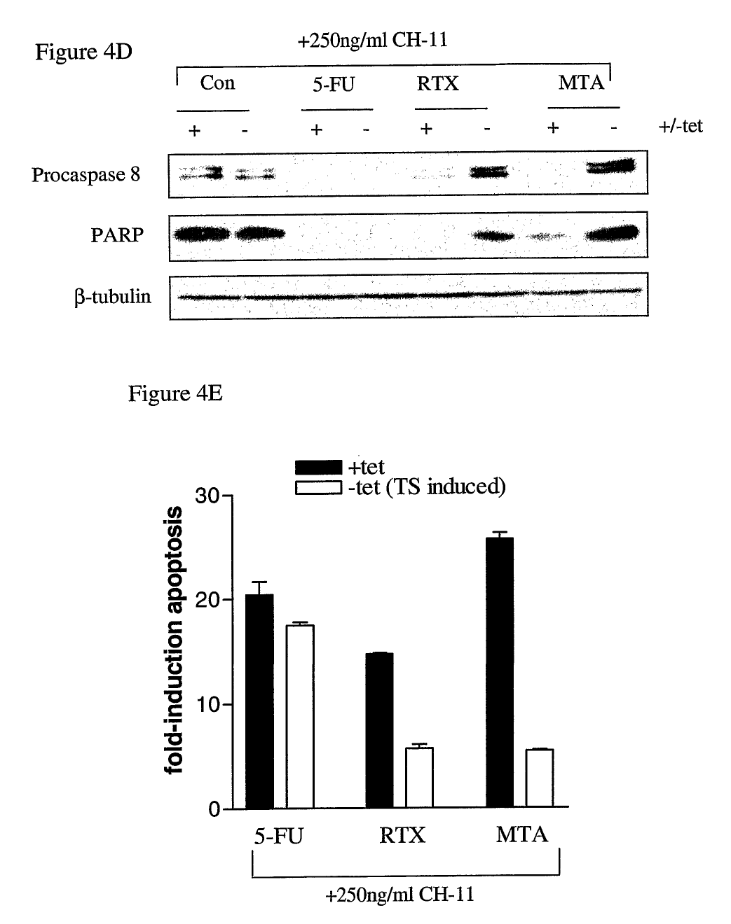

FIG. 4A illustrates tetracycline (tet)-regulated expression of a TS trans-gene in M7TS90 cells.

FIG. 4B illustrates Western blot analysing the effect of TS induction (-tet lanes) on Fas up-regulation in M7TS90 cells in response to treatment with 10 μM 5-FU, 100 nM RTX or 1 μM MTA for 72 hours.

FIG. 4C illustrates an MTT assay showing the impact of TS induction (-tet) on viability of M7TS90 cells following treatment with 5-FU (10 μM) or RTX (100 nM) in the presence of co-treatment with 250 ng/ml CH-11.

FIG. 4D illustrates the impact of TS induction on caspase 8 activation and processing of full-length (118 kDa) PARP in M7TS90 cells treated with 5-FU (10 μM), RTX (100 nM) or MTA (1 μM) followed by 250 ng/ml CH-11.

FIG. 4E illustrates Effect of exogenous TS expression on the induction of apoptosis in M7TS90 cells treated with 5-FU (10 μM) RTX (100 nM) or MTA (1 μM) in the presence of co-treatment with 250 ng/ml CH-11. Apoptosis was assessed by analysing the sub-G₁/G₀ fraction of propidium iodide stained cells by flow cytometry. Equal loading of Western blots was assessed by analysing β-tubulin expression. For each combined treatment the cells were pre-treated with chemotherapeutic drug for 72 hours followed by CH-11 for a further 24 hours.

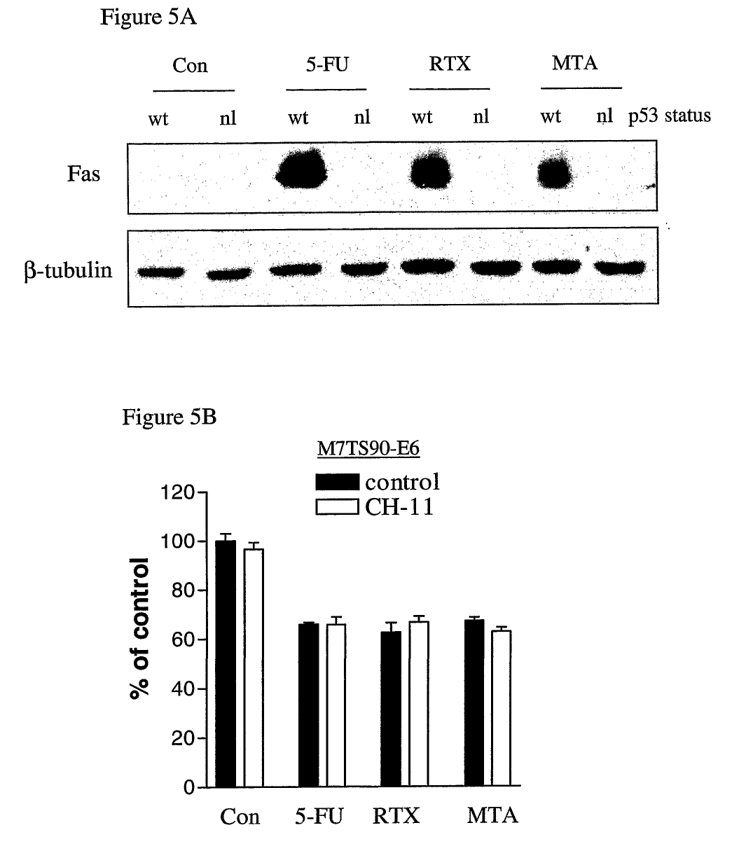

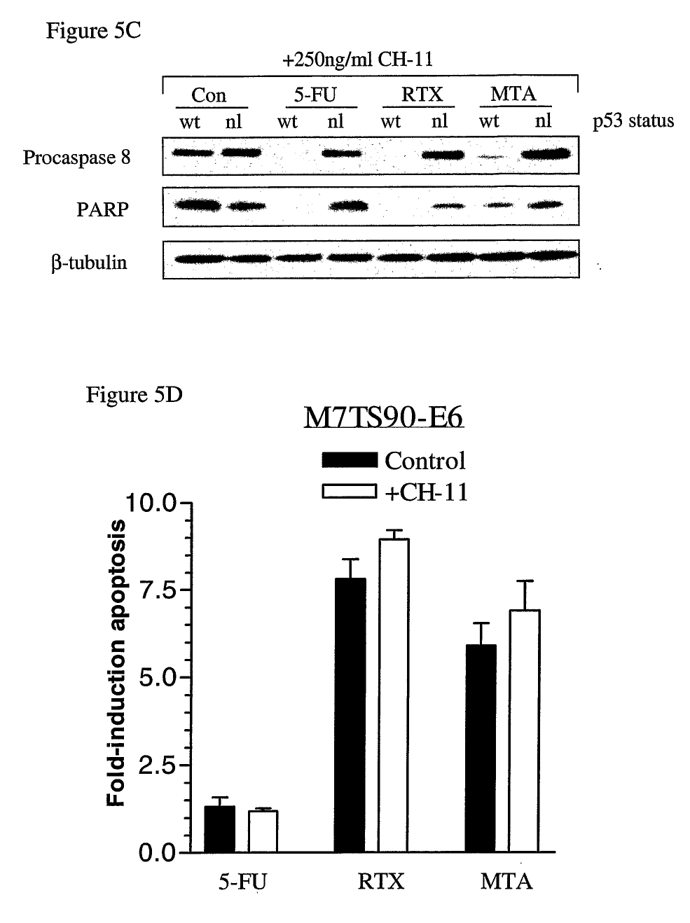

FIG. 5A illustrates Western blot analysis of Fas expression in p53 wild type (wt) M7TS90 and p53 null (n1) M7TS90-E6 cells 72 hours after treatment with no drug (Con), 10 μM 5-FU, 100 nM RTX or 1 μM MTA.

FIG. 5B illustrates MTT cell viability assays in p53 null M7TS90-E6 cells treated with 10 μM 5-FU, 100 nM RTX or 1 μM MTA in combination with 250 ng/ml CH-11.

FIG. 5C illustrates Western blot analysis of procaspase 8 and full-length (118 kDa) PARP expression in M7TS90 (wt) and M7TS90-E6 (n1) cells treated with 5-FU (10 μM), RTX (100 nM) or MTA (1 μM) followed by 250 ng/ml CH-11.

FIG. 5D illustrates Effect of CH-11 (250 ng/ml) on the induction of apoptosis in M7TS90-E6 cells treated with 5-FU (10 μM) RTX (100 nM) or MTA (1 μM). Apoptosis was assessed by analysing the sub-G₁/G₀ fraction of propidium iodide stained cells by flow cytometry. Equal loading of Western blots was assessed by analysing β-tubulin expression. For each combined treatment the cells were pre-treated with chemotherapeutic drug for 72 hours followed by CH-11 for a further 24 hours.

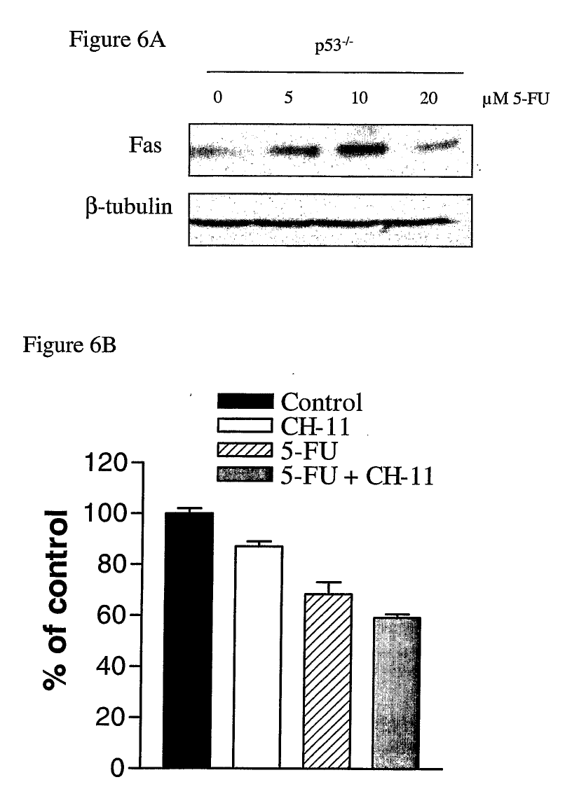

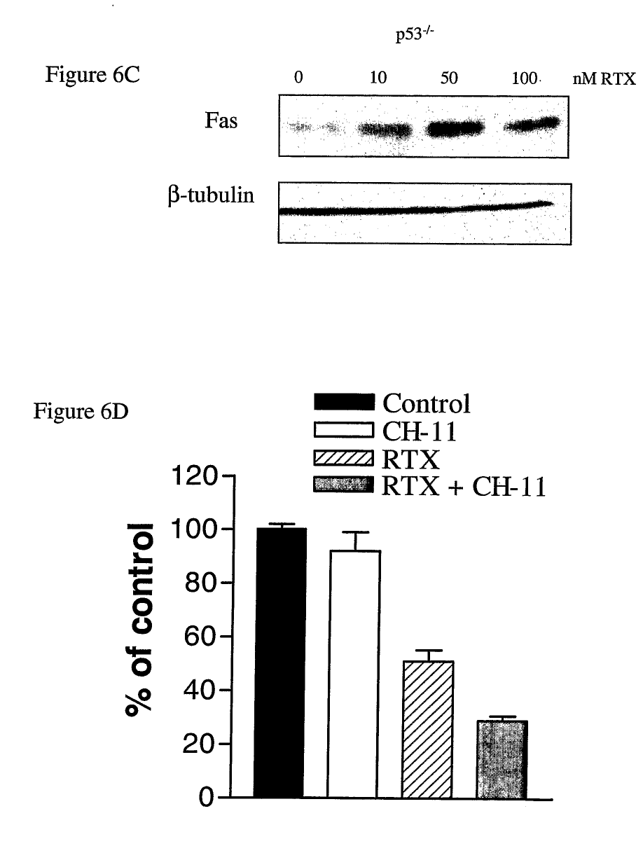

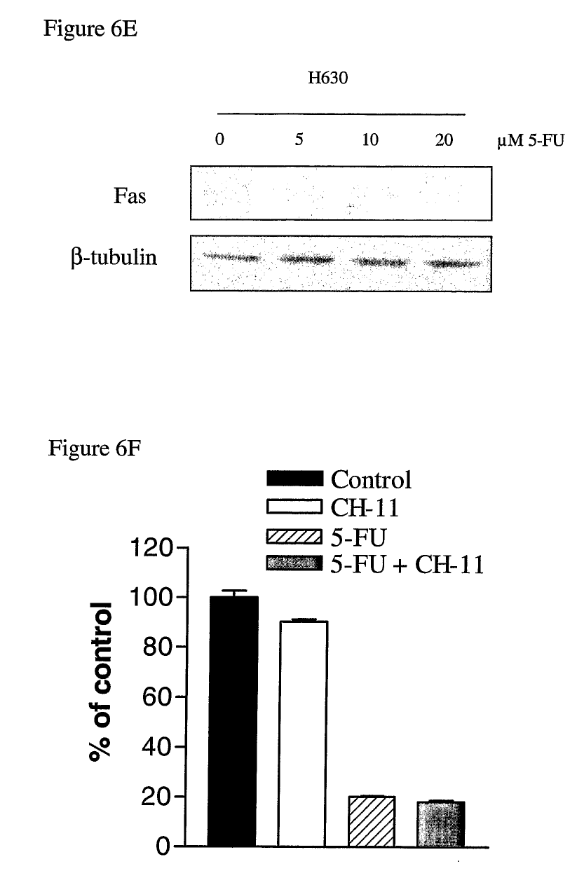

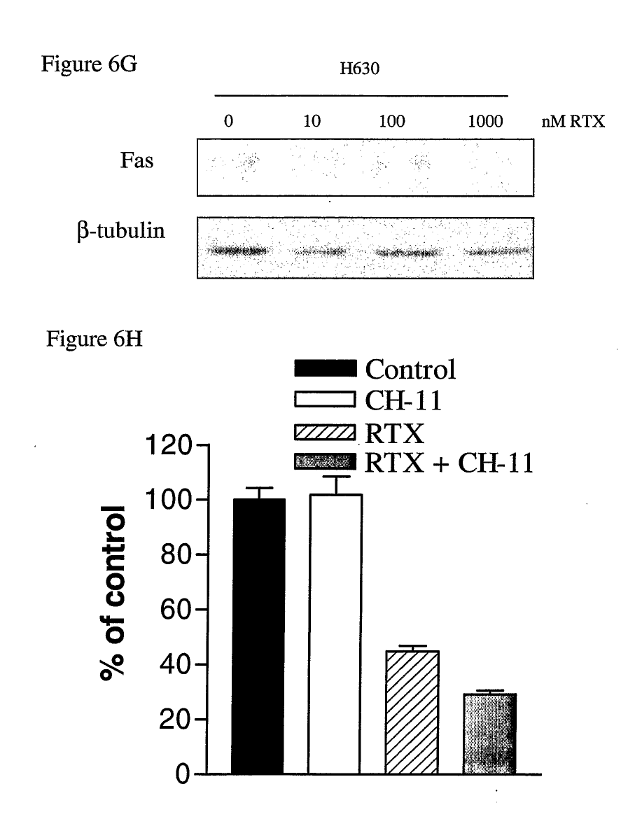

FIG. 6A illustrates Western blot analysis of Fas expression in HCT116p53^{-/-} cells treated with a range of concentrations of 5-FU for 48 hours.

FIG. 6B illustrates MTT cell viability assays in HCT116p53^{-/-} cells treated with no drug (control), CH-11 alone (250 ng/ml), 5-FU alone (10 μM), or co-treated with 5-FU and CH-11. The decrease in cell viability for the combined treatment was not synergistic (RI=1.01).

FIG. 6C illustrates Western blot analysis of Fas expression in HCT116p53^{-/-} cells treated with a range of concentrations of RTX for 48 hours.

FIG. 6D illustrates MTT cell viability assays in HCT116p53^{-/-} cells treated with no drug (control), CH-11 alone (250 ng/ml), RTX alone (50 nM), or co-treated with RTX and CH-11. The decrease in cell viability for the combined treatment was synergistic (RI=1.62, p=0.01).

FIG. 6E illustrates Western blot analysis of Fas expression in H630 cells treated with a range of concentrations of 5-FU for 48 hours.

FIG. 6F illustrates MTT cell viability assays in H630 cells treated with no drug (control), CH-11 alone (250 ng/ml), 5-FU alone (10 μM), or co-treated with 5-FU and CH-11. The decrease in cell viability for the combined treatment was not synergistic (RI=0.99).

FIG. 6G illustrates Western blot analysis of H630 cells treated with a range of concentrations of RTX for 48 hours.

FIG. 6H illustrates MTT cell viability assays in H630 cells treated with no drug (control), CH-11 alone (250 ng/ml), RTX alone (50 nM), or co-treated with 5-FU and CH-11. The decrease in cell viability for the combined treatment was synergistic (RI=1.41, p<0.005). Equal loading of Western blots was assessed by analysing β-tubulin expression. For each combined treatment the cells were pre-treated with chemotherapeutic drug for 72 hours followed by CH-11 for a further 24 hours.

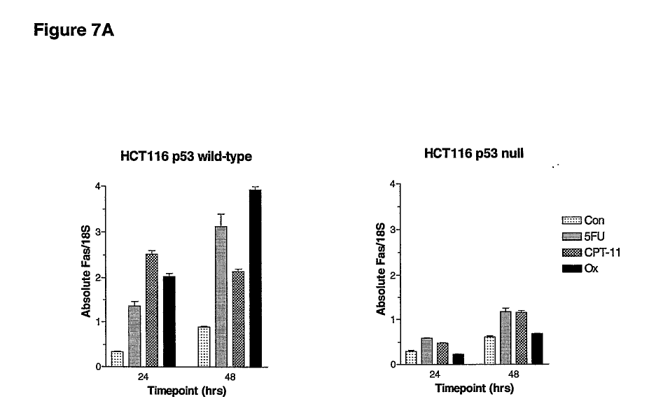

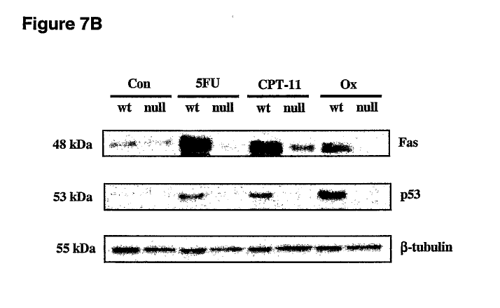

FIG. 7 illustrates FIG. 7 A, Expression of Fas/CD95 mRNA by real-time PCR in the isogenic HCT116 p53 wild-type and null cell lines following treatment with 5-fluorouracil (5-FU), CPT-11 and Oxaliplatin for 24 and 48 hours. Gene expression was calculated at each timepoint as a ratio of the target gene Fas to 18S. The expression of each gene was calculated according to standard curves generated for each gene using a dilution series. B, Western blot analysis of Fas and p53 expression in the HCT116 p53 wild-type and null cell lines following treatment with 5-FU 5 μM, CPT-11 5 μM and Oxaliplatin 1 μM for 24 hours.

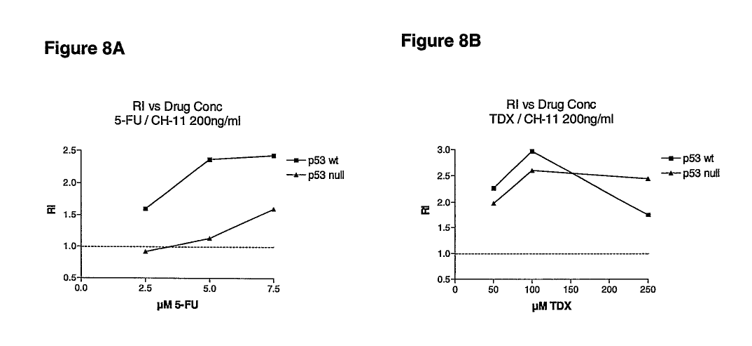

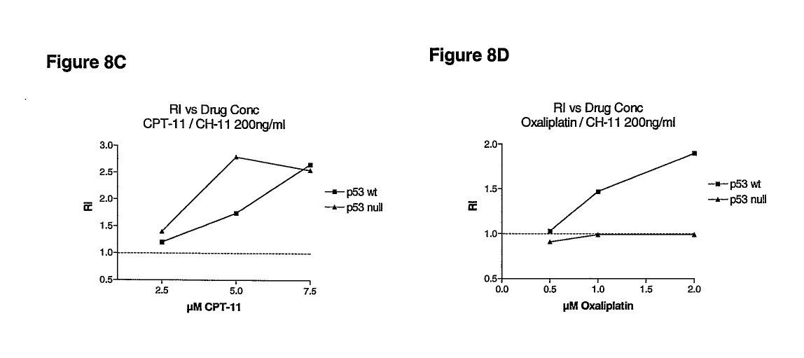

FIG. 8 illustrates graphs of RI values calculated from MTT cell viability assays of the chemotherapeutic agents 5-FU, Tomudex (TDX), CPT-11 and Oxaliplatin used in combination with the agonistic anti-Fas antibody CH-11 (200 ng/ml). The RI is calculated as ratio of the expected cell survival (Sexp, defined as the product of the survival observed with drug A alone and the survival observed with drug B alone) to the observed cell survival (Sobs) for the combination of A and B (RI=Sexp/Sobs). Synergism is defined as an RI greater than 1.

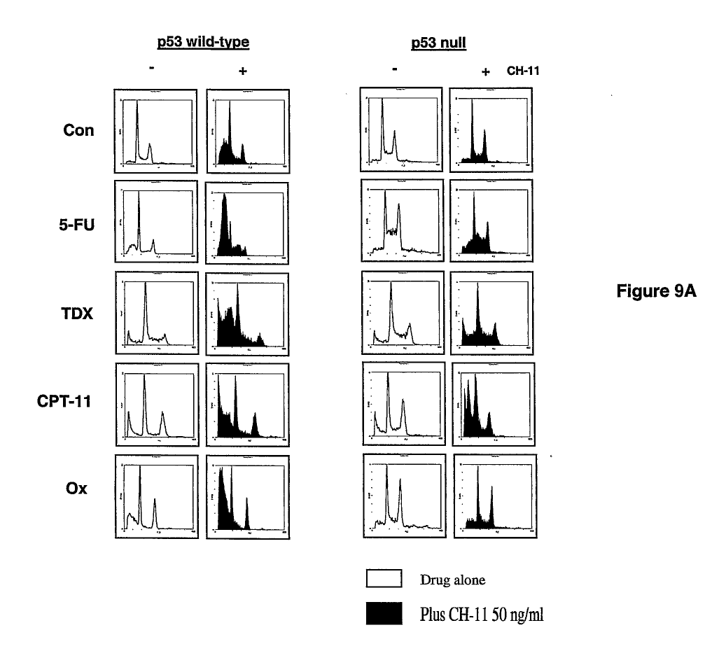

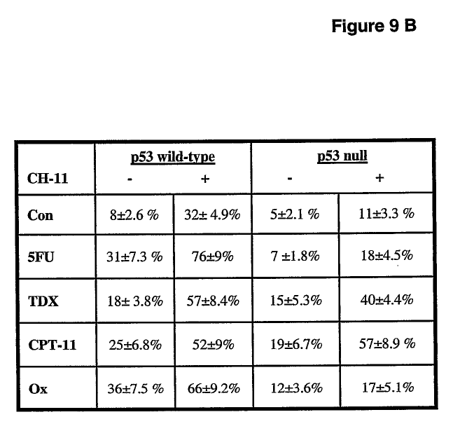

FIG. 9 illustrates A, Flow cytometry analysis of cells stained with propidium iodide stained HCT116 p53 wild-type and null cells treated with 5-FU (5 μM), TDX (50 nM), CPT-11 (5 μM) and Oxaliplatin (1 μM) for 24 hours and then with CH-11 (50 ng/ml) for an additional 24 hours. B, Sub G0/G1 populations for the HCT116p53 wild-type and null cell lines treated with chemotherapy drugs with and without CH-11 50 ng/ml.

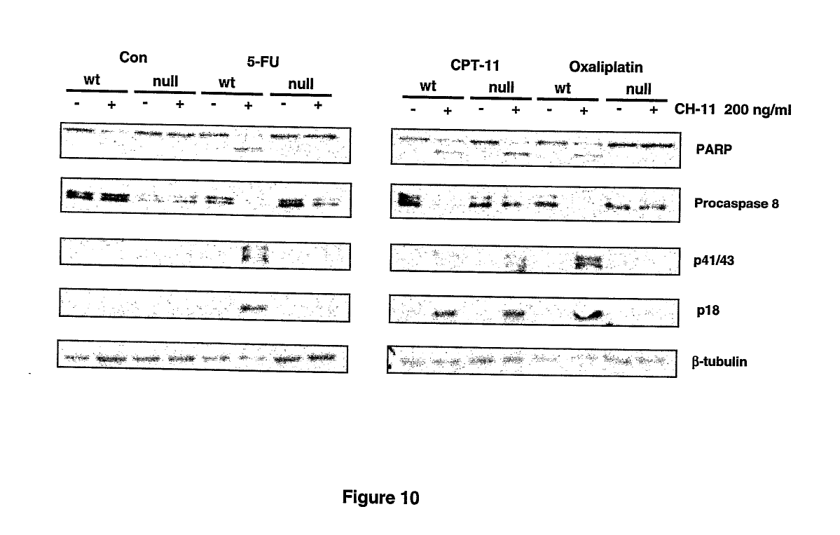

FIG. 10 illustrates the effect of adding CH-11 200 ng/ml for 24 hours to HCT116 p53 wild-type and null cells already treated for 24 hours with 5-FU (5 μM), CPT-11 (5 μM) and Oxaliplatin (1 μM) on PARP cleavage and activation of procaspase 8 by Western blot analysis.

FIGS. 11A and 11B illustrates Fas cell surface expression in HCT116 p53 wild-type and null cell lines treated with chemotherapeutic agents.

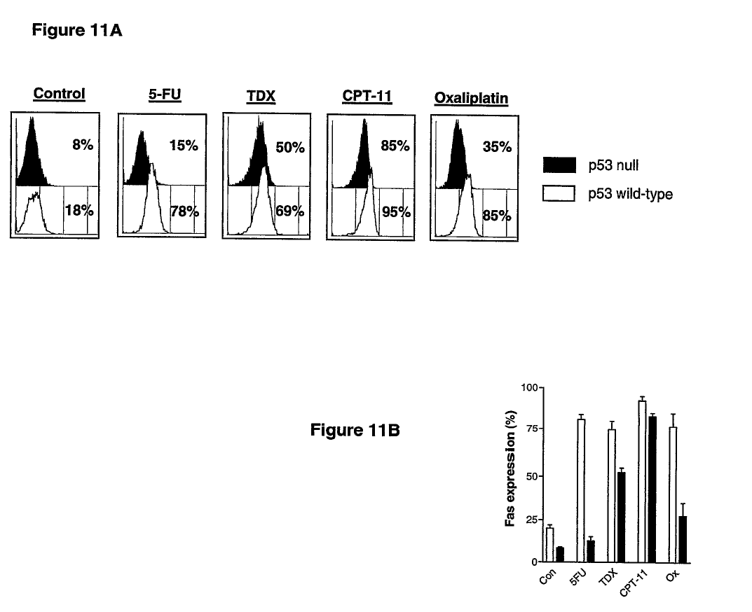

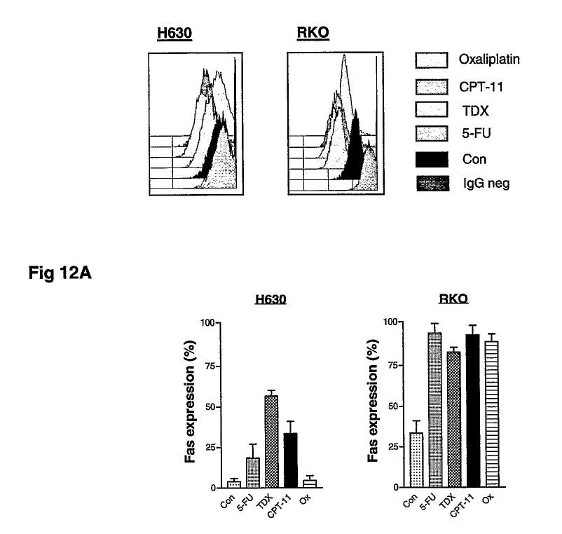

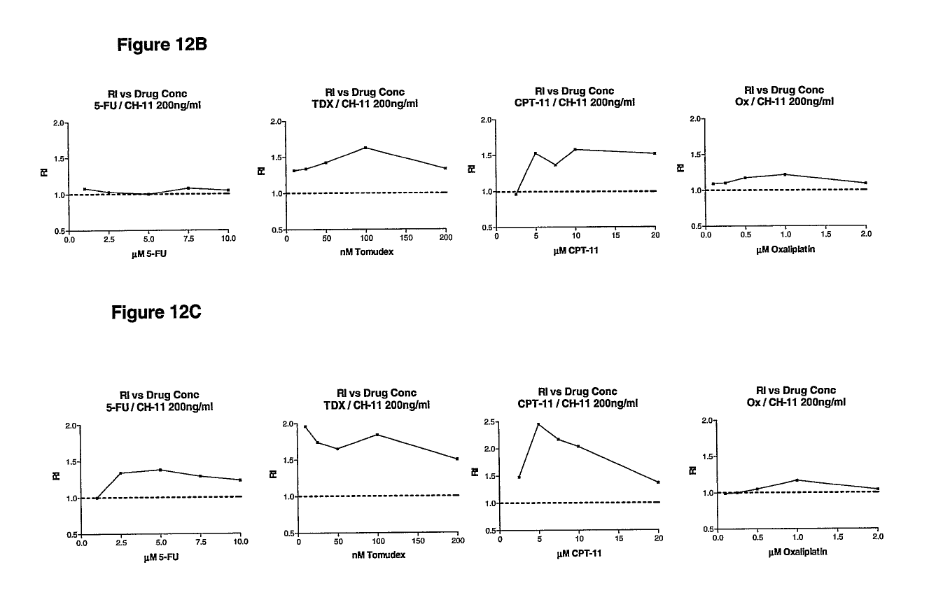

FIG. 12 illustrates A, Fas expression in the H630 and RKO cell lines treated with 5-FU (5 μM), CPT-11 (5 μM) and Oxaliplatin (1 μM) for 48 hours. B,C RI values calculated from MTT cell viability assays of the chemotherapeutic agents 5-FU, CPT-11 and Oxaliplatin used in combination with the agonistic anti-Fas antibody CH-11 (200 ng/ml) in the H630 (B) and RKO (C) cell lines.

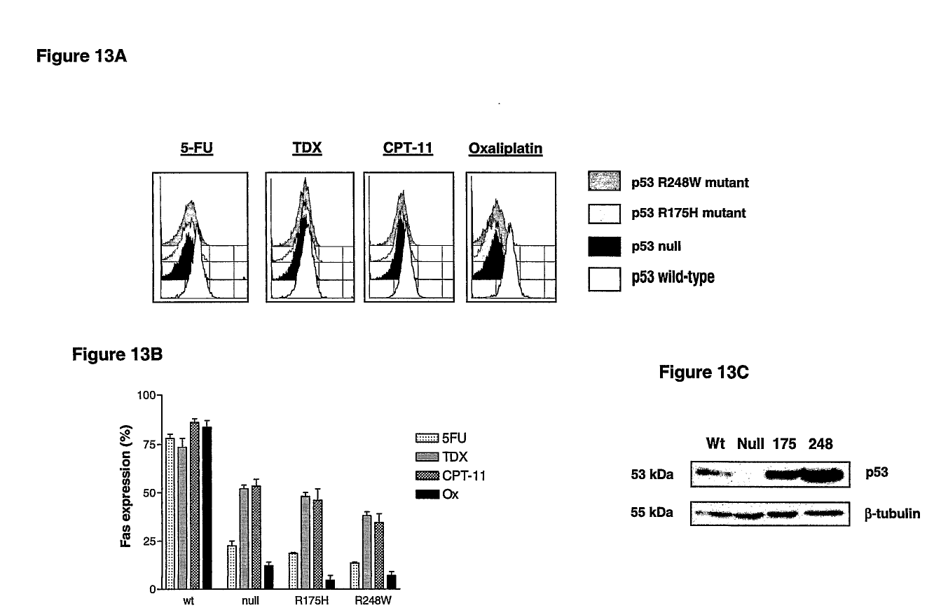

FIG. 13 illustrates A, Analysis for Fas-expressing cells in HCT116 p53 wild-type, null and the R175H and R248W p53 mutant cell lines. B, The percentage of cells expressing the Fas death receptor (5000 cells were examined for each sample). C, p53 protein expression in HCT116 p53 wild-type, null, R175H and R248W mutant cell lines. Cells were harvested after treatment for flow cytometric analysis.

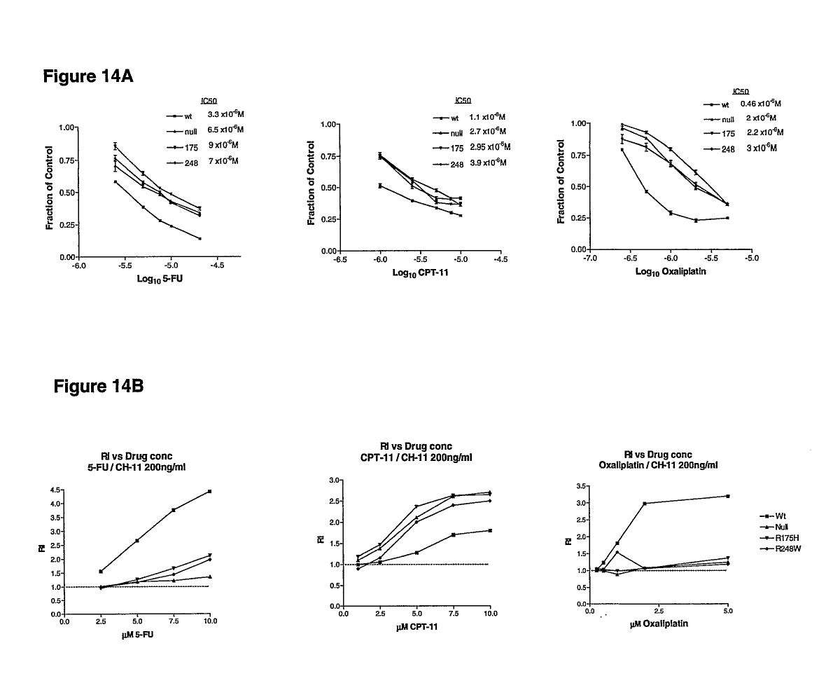

FIG. 14 illustrates A, MTT assays of HCT116 p53 wild-type and null cell lines together with R175H and R248W p53 mutants treated with IC60 doses of 5-FU, CPT-11 and Oxaliplatin at 72 hrs. B, RI values calculated from MTT cell viability assays of the chemotherapeutic agents 5-FU, CPT-11 and Oxaliplatin used in combination with the agonistic anti-Fas antibody CH-11 (200 ng/ml) in the p53 wild-type and null cell lines and the R175H and R248W p53 mutant lines.

### MATERIALS AND METHODS

Cell Culture. All cells were maintained in 5% CO₂ at 37° C. MCF-7, H630 (p53 mutation in exon 10) and RKO (wild-type p53) cells were maintained in DMEM with 10% dialyzed bovine calf serum supplemented with 1 mM sodium pyruvate, 2 mM L-glutamine and 50 μg/ml penicillin/streptomycin (from Life Technologies Inc., Paisley, Scotland). M7TS90 cells (6) were maintained in ‘MCF-7 medium’ supplemented with 1 μg/ml puromycin, 1 μg/ml tetracycline (from Sigma, Poole, Dorset, England), and 100 μg/ml G418 (from Life Technologies Inc). M7TS90-E6 cells (6) were maintained in ‘M7TS90 medium’ supplemented with 200 μg/ml hygromycin (Life Technologies Inc). To induce expression of exogenous TS, cells were washed three times in 1×PBS and incubated in growth medium lacking tetracycline. HCT116 p53^{+/+} and p53^{-/-} isogenic human colon cancer cells were kindly provided by Professor Bert Vogelstein (John Hopkins University, Baltimore, Md.). HCT116 cell lines were grown in McCoy's 5A medium (GIBCO) supplemented with 10% dialysed foetal calf serum, 50 μg/ml penicillin-streptomycin, 2 mM L-glutamine and 1 mM sodium pyruvate.

p53 Mutant Cell Lines

Plasmids containing the p53 mutations R175H and R248W were kindly provided by Prof. G. Lozano (MD Anderson Cancer Center, Houston). The R175H mutation is at codon 175 of the p53 gene and results in an amino acid change of arginine to histidine. The R248W mutation is at codon 248 and results in an amino acid change of arginine to tryptophan. p53 248 and 273 mutant HCT116 cell lines were created by dual transfection of HCT116 p53 null cells with a plasmid containing the appropriate mutant p53 gene (pC53NN 248/273 mutant, kind gift of Guilermina Lozano, University of Texas MD Anderson Cancer Center) and a plasmid containing a puromycin resistance gene (pIRESpuro3, BD Biosciences, CA, USA). Cells were seeded on p90 dishes in Optimem, (Invitrogen) supplemented with 10% FCS, at a density of 1×10⁵ cells/dish. Transfection was carried out the following day when the monolayer was approximately 70% confluent. Cells were cotransfected with 1 μg pIRESpuro3 DNA and 20 μg pC53NN p53 mutant DNA using Genejuice Transfection reagent (Novagen, CA, USA) according to the manufacturer's instructions. Stably transfected cells were selected over approximately 14 days in medium containing 1 g/ml puromycin. Colonies were harvested and assayed for mutant p53 expression by Western blot using an anti-p53 mAb (DO-1, Santa Cruz Biotechnology, CA, USA) and also by nucleotide sequencing. The DO-1 antibody binds to an N-terminal epitope between amino acid residues 11 and 25.

Northern blot analysis. Northern blots were performed as described previously using a cDNA probe complementary to the Fas coding region (7). Equal loading was assessed using a P-tubulin cDNA probe.

Western Blotting. Western blots were performed as previously described (6). The Fas/CD95, Bc1-2 and BID (Santa Cruz Biotechnology, Santa Cruz, Calif.), caspase 8 (Oncogene Research Products, Darmstadt, Germany), p53 (Santa Cruz Biotechnology), and PARP (Pharmingen, BD Biosciences, Oxford, England) mouse monoclonal antibodies were used in conjunction with a horseradish peroxidase (HRP)-conjugated sheep anti-mouse secondary antibody (Amersham, Little Chalfont, Buckinghamshire, England). FasL rabbit polyclonal antibody (Santa Cruz Biotechnology) was used in conjunction with an HRP-conjugated donkey anti-rabbit secondary antibody (Amersham). TS sheep monoclonal primary antibody (Rockland, Gilbertsville, Pa.) was used in conjunction with an HRP-conjugated donkey anti-sheep secondary antibody (Serotech, Oxford, England). Equal loading was assessed using a β-tubulin mouse monoclonal primary antibody (Sigma).

Cell Viability Assays. Cell viability was assessed by MTT (3-(4,5-dimethylthiazol-2-yl)-2,5-diphenyltetrazolium bromide, Sigma) assay (12). Where cell viability assays were employed in Examples 1 to 5, the protocol was as follows. Cells were seeded at 2,500 cells per well on 96-well plates 24 hours prior to drug treatment and then treated with a range of concentrations of 5-FU, RTX and MTA for 72 hours, following which time the agonistic Fas monoclonal antibody, CH-11 (MBL, Watertown, Mass.), was added (10-250 ng/ml) for a further 24 hours. MTT (0.5 mg/ml) was then added to each well and the cells incubated at 37° C. for a further 3 hours. The culture medium was removed and formazan crystals reabsorbed in 200 μL DMSO. Cell viability was determined by reading the absorbance of each well at 570 nm using a 96-well microplate reader (Molecular Devices, Wokingham, England). Where cell viability assays were employed in Examples 6 to 12, the protocol was as follows. Cells were seeded at 1800 cells per well on 96-well plates 24 hours prior to drug treatment. Cells were treated with a range of concentrations of 5-FU (Faulding Pharmaceuticals, UK), TDX (AstraZeneca, UK), CPT-11 (Aventis, UK) and Oxaliplatin (Sanofi-Synthelabo, UK) for 24 hours, following which time the agonistic Fas monoclonal antibody, CH-11 (MBL), was added (200 ng/ml) for 48 hours. Twenty microlitres (20 μL) MTT (5 mg/ml) was then added to each well and the cells incubated at 37° C. for a further 3 hours. The culture medium was removed and formazan crystals reabsorbed in 200 μL DMSO. Cell viability was determined by reading the absorbance of each well at 570 nm using an ELISA plate reader (Molecular Devices).

Flow Cytometric Analysis.

Where flow cytometric analysis was employed in Examples 1 to 5, the following protocol was used. Cells were seeded at 1×10⁵ per well of a 6-well tissue culture plate. After 24 hours, 5-FU, RTX or MTA were added to the medium and the cells cultured for a further 72 hours, after which time 250 ng/ml CH-11 was added for 24 hours. DNA content of harvested cells was evaluated after propidium iodide staining of cells using the EPICS XL Flow Cytometer (Coulter, Miami, Fla.). Where flow cytometric analysis was employed in Examples 6 to 12, the following protocol was used. Cells were seeded at 1×10⁵ per well of a 6-well tissue culture plate. After 24 hours, 5-FU, TDX, CPT-11 or oxaliplatin were added to the medium and the cells cultured for a further 24 hours, after which time 50 ng/ml CH-11 was added for 24 hours. DNA content of harvested cells was evaluated after propidium iodide staining of cells using the EPICS XL Flow Cytometer (Coulter). A FITC-conjugated monoclonal anti-human Fas antibody (DAKO) was used to determine cell surface expression of the receptor. Cells were collected and washed twice in PBS before incubating in the antibody (1/20 dilution) at 4° C. for 30 mins. A non-reactive FITC-conjugated mouse IgG1 antibody was used as a negative control for each sample. Following incubation the cells were washed twice with PBS containing 2% bovine serum albumin and fixed in 0.3 mL 4% paraformaldehyde. Samples were then analysed on the EPICS XL Flow Cytometer (Coulter).

Real-Time PCR

Real-time PCR was performed using a DNA Engine Opticon 2 (MJ Research Incorporated). TheDyNAmo SYBR Green qPCR kit (Finnzymes) was used as the fluoresent dye specific for double-stranded DNA. PCR conditions consisted of an initial denaturation step of 95° C. for 10 minutes, followed by 39 cycles of 94° C. for 10 secs, 55° C. for 20 secs and 72° C. for 20 secs, with a final extension of 72° C. for 10 minutes. A melting curve was included at the end of each run to check the specificity of the amplified product. Experiments were performed in triplicate to ensure reproducibility of the technique. On completion of the run the PCR products were run put on a 2% ethidium bromide agarose gel to confirm that their size matched that of the expected amplicon. Primer sequences were as follows: Fas (Forward) AAAGGCTTTGTTCGAAAG, Fas (Reverse) CACTCTAGACCAAGCTTTGG, 18S (Forward) CATTCGTATTGCGCCGCTA, 18S (Reverse) CGACGGTATCTGATCGTCT.

Statistical Analyses. The nature of the interaction between the chemotherapeutic drugs and CH-11 was determined by calculating the R index (RI), which was initially described by Kern and later modified by Romaneli (13, 14). The RI is calculated as the ratio of expected cell survival (Sₑₓₚ, defined as the product of the survival observed with drug A alone and the survival observed with drug B alone) to the observed cell survival (S_{obs}) for the combination of A and B (RI=Sₑₓₚ/S_{obs}). Synergism is then defined as an RI of greater than unity. Romanelli et al. suggest that a synergistic interaction may be of pharmacological interest when RI values are around 2.0 (14). This method was selected because treatment with CH-11 alone had little effect on cell viability, which meant that other methods such as the median effect principle (15) and isobologram methods were not suitable (16). To further assess the statistical significance of the interactions, the inventors designed a univariate ANOVA analysis using the SPSS software package. This was an additive model based on the null hypothesis that there was no interaction between the drugs.

Results

### EXAMPLE 1

### Fas is Highly Up-Regulated in Response to 5-FU and RTX

Using DNA microarray profiling, the inventors previously identified the Fas death receptor as being highly up-regulated in response to 5-FU in MCF-7 cells (7). Northern blot analyses confirmed that Fas mRNA was up-regulated in MCF-7 cells 48 hours following treatment with an IC₆₀ dose (5 μM) of 5-FU (FIG. 1A). Analysis of Fas protein expression in MCF-7 cells revealed that it was up-regulated by ˜12-fold 72 hours after treatment with 5-FU (FIG. 1B). Fas was also highly up-regulated (by ˜7-fold) in response to treatment with an IC₆₀ dose (25 nM) of RTX (FIG. 1B).

### EXAMPLE 2

### The Agonistic Fas Monoclonal Antibody CH-11 Synergistically Activates Apoptosis in Response to 5-FU and RTX

To examine the role of the Fas signalling pathway in mediating the response of MCF-7 cells to 5-FU and RTX, the inventors used the agonistic Fas monoclonal antibody CH-11. Cells were treated with IC₆₀ doses of each drug for 72 hours, after which time they were treated with 250 ng/ml CH-11 for a further 24 hours. Treatment with 5 μM 5-FU alone resulted in a ˜60% reduction in cell viability compared to control (FIG. 1C). Treatment with CH-11 alone without prior incubation with 5-FU caused a modest ˜6% decrease in cell viability. However, treatment with 5-FU followed by CH-11 was found to result in an ˜84% decrease in cell viability. The combined treatment had an RI value of 2.40 indicating that the interaction was highly synergistic. This was further confirmed by ANOVA analysis, which indicated that the synergistic interaction between the drugs was highly statistically significant (p<0.0005). Similarly, treatment with 25 nM RTX for 72 hours followed by CH-11 for 24 hours produced a highly synergistic decrease in cell viability (RI=2.22, p<0.0005, FIG. 1D). An IgM isotype control antibody had no effect on the cell viability of drug-treated cells (data not shown).

To assess the degree of apoptosis in MCF-7 cells treated with 5-FU and RTX individually, or in combination with CH-11, the inventors carried out flow cytometry of propidium iodide stained cells and analysed the sub-G₁/G₀ apoptotic fraction. Cells were treated with either 5-FU or RTX for 72 hours followed by 250 ng/ml CH-11 treatment for 24 hours. The inventors found that CH-11 alone had little effect on apoptosis (FIGS. 1E and F). Treatment with 5-FU alone for 96 hours resulted in a modest ˜2-fold induction of apoptosis in response to 5 μM 5-FU (FIG. 1E). However, addition of CH-11 to 5-FU-treated cells resulted in a dramatic increase in apoptosis, with a ˜12-fold induction of apoptosis following co-treatment with 5 μM 5-FU and CH-11. Similarly, the combination of RTX with CH-11 resulted in dramatic activation of apoptosis, with 60% of cells in the sub-G₁/G₀ apoptotic phase following combined treatment with 25 nM RTX and CH-11 compared to ˜11% in untreated control cells, ˜16% in cells treated with RTX alone and ˜18% in cells treated with CH-11 alone (FIG. 1F). The activation of apoptosis by CH-11 in 5-FU and RTX treated cultures was observed across a range of concentrations of each drug (FIGS. 1E and F), indicating that the synergistic interaction between CH-11 and both drugs was due to activation of apoptosis.

The inventors next examined the ability of CH-11 to activate apoptosis in other cell lines. Treatment of HCT116p53^{+/+} colon cancer cells with 5-FU resulted in potent up-regulation (>10-fold) of Fas expression after 48 hours (FIG. 2A). Furthermore, treatment with 5 μM 5-FU followed by 250 ng/ml CH-11 synergistically decreased cell viability in this line with an RI value of 1.92 (p<0.005). Similarly, RTX treatment dramatically increased Fas expression after 72 hours (FIG. 2C), while treatment with RTX followed by CH-11 resulted in a highly synergistic decrease in cell viability (FIG. 2D, RI=3.44, p<0.0005). The inventors also examined another p53 wild type colon cancer cell line, RKO. As was the case with both MCF-7 and HCT116p53^{+/+} cells, both 5-FU and RTX treatment resulted in dramatic Fas up-regulation 48 hours post-treatment (FIGS. 3E and F). Furthermore, treatment of RKO cells with 5-FU or RTX followed by CH-11 synergistically decreased cell viability with RI values of 1.74 (p<0.0005) and 2.31 (p<0.0005) respectively (FIGS. 3F and G). These results indicate that CH-11 not only activates apoptosis of 5-FU- and RTX-treated MCF-7 breast cancer cells, but also of HCT116p53^{+/+} and RKO colon cancer cells. The inventors also found that treatment with the antifolate MTA up-regulated Fas expression and synergistically interacted with CH-11 to decrease cell viability in all three cell lines (data not shown).

### EXAMPLE 3

### Effect of 5-FU and RTX on Fas Signal Transduction

The inventors next examined drug-induced activation of the Fas signalling pathway in response to 5-FU and RTX. Although Fas was highly up-regulated (>10-fold) in MCF-7 cells in response to IC₆₀ doses of either drug, FasL expression was unaffected (FIG. 3A). Surprisingly, neither caspase 8, nor its substrate BID were activated in 5-FU- or RTX-treated cells as indicated by a lack of down-regulation of the levels of procaspase 8 or full-length BID (FIG. 3A). The inventors subsequently analysed activation of the Fas pathway in MCF-7 cells following co-treatment with 5-FU and CH-11. Fas, procaspase 8 and BID expression levels were determined in cells treated with 5 μM 5-FU for 72 hours followed by 250 ng/ml CH-11 for 24 hours and compared to cells treated with 5-FU alone or CH-11 alone for the appropriate time periods (FIG. 3B). Treatment with CH-11 alone had no effect on Fas, procaspase 8 or BID expression (FIG. 3B, lane 2). As already noted, treatment with 5-FU alone resulted in dramatic up-regulation of Fas, but had no effect on procaspase 8 or BID expression, indicating that neither molecule was activated (FIG. 3B, lane 3). However, treatment of MCF-7 cells with 5-FU and CH-11 resulted in a dramatic activation of both caspase 8 and BID as indicated by complete loss of procaspase 8 and full-length BID expression in these cells (FIG. 3B, lane 4). Similarly, in HCT116p53^{+/+} cells activation of caspase 8 was only observed following co-treatment with either 5-FU and CH-11 or RTX and CH-11 (FIG. 3C). Furthermore, cleavage of PARP (poly(ADP) ribose polymerase), a hallmark of apoptosis, was only observed in HCT116p53^{+/+} cells co-treated with each drug and CH-11.

The inventors next compared the kinetics of caspase 8 activation with cleavage of PARP. Six hours after addition of CH-11 to MCF-7 cells pre-treated for 72 hours with 5 μM 5-FU, procaspase 8 levels were reduced by ˜3-fold compared to time zero (FIG. 3D). This coincided with PARP cleavage, which is indicative of cells undergoing apoptosis. Thus, activation of caspase 8 coincided with the onset of apoptosis. Twelve and 18 hours following CH-11 treatment, the levels of procaspase 8 had fallen to less than 5% of that observed at time zero, indicating potent activation of caspase 8. The inventors further examined the relationship between caspase 8 activation and apoptosis using the specific caspase 8 inhibitor IETD-fmk. Cells were pre-treated with 5 μM 5-FU for 72 hours followed by 250 ng/ml CH-11, 10 μM IETD-fmk, or a combination of CH-11 and IETD-fmk for 24 hours. Fas was highly up-regulated in all treatment groups (FIG. 3D). As noted above, the combination of 5-FU and CH-11 resulted in a dramatic activation of caspase 8 and PARP cleavage (FIG. 3E, lane 2). Addition of the caspase 8 inhibitor had no effect on protein expression in cells treated with 5-FU alone (FIG. 3E, lane 3). However, IETD-fmk blocked processing of procaspase 8 in cells co-treated with 5-FU and CH-11 (FIG. 3E, lane 4). This result indicates that caspase 8 activity is necessary for procaspase 8 processing at the DISC and is consistent with the induced proximity model proposed for caspase 8 activation (17). Significantly, blocking caspase 8 activation also inhibited PARP cleavage in 5-FU/CH-11 co-treated cells, indicating that apoptosis of these cells is dependent on caspase 8 activation.

### EXAMPLE 4

### Effect of TS Induction on the Synergy Between CH-11 and 5-FU, RTX and MTA

Treatment with 5-FU and TS-targeted antifolates has been shown to acutely increase TS expression, most likely through disruption of a negative feedback mechanism in which TS binds to and inhibits translation of its own mRNA (18). This constitutes a potential mechanism of resistance as TS induction would facilitate recovery of enzymatic activity. The inventors therefore examined the effect of inducible TS expression on 5-FU and antifolate-mediated up-regulation of Fas and the synergistic interaction between CH-11 and each drug. To do this, the inventors used the MCF-7-derived M7TS90 cell line (6), in which transcription of a TS trans-gene is activated following withdrawal of tetracycline (tet) from the culture medium (FIG. 4A). In agreement with the inventors previous findings, TS induction in the M7TS90 cell line abrogated RTX- and MTA-, but not 5-FU-mediated up-regulation of Fas (FIG. 4B) (6). Furthermore, induction of the TS trans-gene had little effect on the synergistic interaction between 5-FU and CH-11 (FIG. 4C). However, TS induction completely abolished the synergistic decrease in cell viability caused by the combination of both 100 nM RTX and CH-11 and 1 μM MTA and CH-11 (FIG. 4C).

The inventors next assessed the effect of inducible TS on caspase 8 activation. The inventors found that TS induction abrogated caspase 8 activation in response to co-treatment with both antifolates and CH-11, but had no effect on caspase 8 activation in response to co-treatment with 5-FU and CH-11 (FIG. 4D). Similarly, TS induction abrogated processing of PARP in cells co-treated with the antifolates and CH-11, but not in cells co-treated with 5-FU and CH-11 (FIG. 4D). The differential effects of TS induction on apoptosis of 5-FU- and antifolate-treated M7TS90 cells was further analysed by flow cytometry by assessing of the sub-G₀/G₁ fraction in cells co-treated with drug and CH-11. Co-treatment with 5-FU and CH-11 resulted in a dramatic ˜20-fold induction of apoptosis in M7TS90 cells that was only modestly reduced to ˜17-fold when TS was induced (FIG. 4E). In contrast, RTX and CH-11 co-treatment resulted in a ˜15-fold increase in the apoptoic fraction, which was reduced to ˜5-fold by TS induction (FIG. 4E). Similarly, combined treatment with MTA and CH-11 resulted in a dramatic ˜26-fold induction of apoptosis that was almost completely abolished by inducible TS expression (FIG. 4E). These results indicate that the activation of Fas-mediated apoptosis in antifolate-treated cells was highly dependent on TS expression levels. In contrast, the 5-FU/CH-11 interaction was relatively insensitive to TS induction in this cell line, suggesting that non-TS-directed effects were primarily responsible for 5-FU cytotoxicity in these cells.

### EXAMPLE 5

### Effect of p53 Inactivation on the Synergy Between CH-11 and 5-FU, RTX and MTA

The inventors next examined the role of p53 in the observed synergy between CH-11 and each drug. p53 has been reported to be an important regulator of Fas expression, both transcriptionally (19) and post-transcriptionally (20). The inventors previously described the generation of p53 null M7TS90-E6 cells by transfection of M7TS90 cells with human papilloma virus (HPV)-E6 (6). Treatment of these p53 null M7TS90-E6 cells with 10 μM 5-FU, 100 nM RTX or 1 μM MTA did not result in Fas up-regulation (FIG. 5A). Furthermore, in contrast to the parental line, the combination of 5-FU and CH-11 did not synergistically decrease cell viability (RI=0.97, FIG. 5B). Similarly, inactivation of p53 also abolished the synergy between RTX and CH-11 and between MTA and CH-11 (RI=0.85 and 1.02 respectively, FIG. 5B).

The inventors further examined the effects of p53 inactivation on drug sensitivity by comparing caspase 8 activation in the p53 wild type and null isogenic M7TS90 lines. Activation of caspase 8 was not observed in the p53 null M7TS90-E6 cells co-treated with each drug and CH-11 (FIG. 5C). In contrast, caspase 8 was potently activated in the parental p53 wild type cell line in response to each co-treatment (FIG. 5C). Inactivation of p53 also completely attenuated PARP cleavage in response to co-treatment with 5-FU and CH-11 (FIG. 5C). However, processing of PARP was evident in p53 null cells treated with both the RTX/CH-11 and MTA/CH-11 combinations, although to a lesser extent than in the p53 wild type line (FIG. 5C). As caspase 8 was not activated, this suggests that antifolate-mediated PARP cleavage in the p53 null cells was not due to activation of Fas-mediated apoptosis by CH-11. Indeed, the inventors found that PARP was also processed in the p53 null cell line in response to treatment with either RTX alone or MTA alone (data not shown). These results indicate that treatment with the antifolates activated p53- and Fas-independent apoptosis in M7TS90-E6 cells. This was further confirmed by flow cytometric analysis of apoptosis in the p53 null cell line. RTX (100 nM) and MTA (1 μM) significantly induced apoptosis of M7TS90-E6 cells by ˜8-fold and ˜6-fold respectively 96 hours after drug treatment (FIG. 5D). In contrast, little apoptosis was observed in M7TS90-E6 cells following treatment with 10 μM 5-FU (FIG. 5D). Importantly, CH-11 had no significant effect on apoptosis induced by any of the drugs in the p53 null cell line.

The inventors extended their studies into the role of p53 in regulating antimetabolite-induced Fas-mediated apoptosis by examining the interaction between these drugs and CH-11 in the p53 null HCT116p53^{-/-} cell line. This cell line was derived from the HCT116p53^{+/+} cell line by somatic knock-out of both p53 alleles (21). Compared to the p53 wild type cell line, there was very little Fas induction in response to 5-FU (FIG. 6A) and RTX (FIG. 6C) in the HCT116p53^{-/-} cell line, with an approximate 2-3-fold induction of Fas expression observed in response to 10 μM 5-FU and 50 nM RTX. Furthermore, no synergistic interaction was observed between 5-FU and CH-11 in the p53 null cell line (RI=1.01, FIG. 6B). Interestingly, a significant synergistic interaction was still observed between RTX and CH-11 in HCT116p53^{-/-} cells (RI=1.62, p=0.01, FIG. 6D), although this was significantly less synergistic than the interaction observed in the p53 wild type parental line (FIG. 2D, RI=3.44, p<0.0005). This suggests that RTX-mediated sensitization of HCT116 cells to CH-11 is not wholly p53-dependent.

The role of p53 in mediating Fas-mediated apoptosis was further examined in the p53 mutant H630 colon cancer cell line. Similar to the p53 null cell lines, Fas was expression was not significantly altered in H630 cells in response to 5-FU (FIG. 6E) or RTX (FIG. 6G). No synergistic decrease in cell viability was observed between 5-FU and CH-11 (FIG. 6F, RI=0.99), however, a statistically significant synergistic interaction was observed between RTX and CH-11 (FIG. 6H, RI=1.64, p<0.0005). This interaction was observed despite the lack of any apparent up-regulation of Fas in response to this agent, suggesting that Fas expression is not the sole determinant of sensitivity to CH-11 in this cell line.

### EXAMPLE 6

### Induction of Fas mRNA by the Chemotherapeutic Agents 5-FU, CPT-11 and Oxaliplatin is p53-Dependent

The majority of studies to date have confirmed that of the Fas/CD95 receptor is p53-dependent. A p53-responsive element has been identified within the first intron of the Fas gene, as well as three putative elements within the promotor [24, 25]. Real-time PCR of the HCT116 p53 wild-type and null cell lines treated with IC60₇₂ₕᵣₛ doses of 5-FU, TDX, CPT-11 and Oxaliplatin for 24 and 48 hours showed significant induction of Fas mRNA expression in response to these agents in the p53 wild-type cells (FIG. 7A). The fold induction of Fas mRNA seen at 24 and 48 hours respectively were 3.8 and 3.4 for 5-FU, 7.0 and 2.5 for CPT-11, and 5.8 and 4.7 for Oxaliplatin. In the HCT116 p53 null cell line treated under similar conditions there was significantly less induction seen, with maximum induction of 2- and 1.9-fold for 5-FU and CPT-11 respectively and no induction seen with Oxaliplatin (FIG. 7A). These results indicate that induction of Fas/CD95 mRNA by these chemotherapeutic agents is p53-dependent.

### EXAMPLE 7

### CPT-11 Treatment Results in a p53-Independent Induction of Fas/CD95 Protein in the HCT116 p53 Null Cell Line

Given that each of the chemotherapeutic agents we examined induced Fas mRNA expression in the HCT116 p53 wild-type cell line flowing treatment, we analysed whether this was reflected as induction of protein expression. Treatment of the HCT116 p53 wild-type and null cell lines with 5-FU 5 μM, CPT-11 5 μM and Oxaliplatin 1 μM for 48 hours resulted in significant induction of Fas/CD95 by all three chemotherapy drugs in the p53 wild-type cell line. The observed induction in this cell line was associated with induction of p53. In contrast, only CPT-11 treatment in the p53 null cell line resulted in induction of Fas/CD95 protein at 48 hours (FIG. 7B).

### EXAMPLE 8

### The Agonistic Fas Monoclonal Antibody CH-11 Synergistically Activates Apoptosis in Response to CPT-11 and TDX in a p53-Independent Manner

The agonistic anti-Fas antibody CH-11 has been shown to activate the Fas/CD95 receptor and cause apoptosis [26]. Lack of up-regulation of the Fas/CD95 receptor in a p53 mutant colon cancer cell line abolished the synergistic interaction between 5-FU and CH-11. In our study treatment of the p53 wild-type and null cell lines with a range of each of the chemotherapy agents 5-FU, TDX, CPT-11 and Oxaliplatin followed 24 hours later by the addition of the anti-Fas antibody CH-11 (200 ng/ml) for a further 48 hours resulted in significant synergy for all the drugs in the p53 wild-type setting, but in the p53 null cells this synergy was only seen with the topoisomerase-I inhibitor CPT-11 and the thymidylate synthase inhibitor TDX. There was no synergistic interaction seen at all with Oxaliplatin in the p53 null cells at any dose, and only slight interaction with 5-FU at the higher doses (FIG. 8). Propidium iodide staining of the HCT116 p53 wild-type and null cell lines treated with these chemotherapeutic agents for 24 hours followed by CH-11 50 ng/ml for an additional 24 hours confirmed that a synergistc interaction is seen with each of the drugs and CH-11 in the p53 wild-type cells (FIG. 9), whereas in the p53 null setting only treatment with CPT-11 and to a lesser extent with TDX resulted in significant synergy with CH-11 50 ng/ml.

### EXAMPLE 9

### Effect of p53 Inactivation on the Synergy Between CH-11 and 5-FU, CPT-11 and Oxaliplatin

Activation of the Fas/CD95 receptor by its natural ligand FasL or the monoclonal antibody CH-11 results in the recruitment and activation of procaspase 8 at the DISC. Procaspase 8 is cleaved to its active subunits p41/43 and p18. Poly(ADP-ribose)polymerase (PARP) is normally involved in DNA repair and stability, and is cleaved by members of the caspase family during early apoptosis.

Western blot analysis of the p53 wild-type and null cell lines treated with IC60 doses of these chemotherapeutic agents for 24 hours followed by a further 24 hours of the anti-Fas antibody CH-11 (200 ng/ml) resulted in PARP cleavage and activation of procaspase 8 (with generation of the active p41/43 and p18 subunits) in the p53 wild-type cell line for each drug (FIG. 10). In the p53 null cell line PARP cleavage and procaspase 8 activation following the addition of CH-11 was only seen following treatment with CPT-11.

### EXAMPLE 10

### CPT-11 Treatment Causes a p53-Independent Induction of Fas/CD95 Cell Surface Expression in the HCT116 p53 Null Cell Line

Flow cytometry demonstrated higher constitutive expression of the Fas/CD95 receptor in the HCT116 p53 wild-type cell line compared to the p53 null cell line. The magnitude of induction of the Fas/CD95 receptor is much higher then would have been predicted from Western blot analysis (FIG. 7B). The ability of TDX and CPT-11 to interact synergistically with the anti-Fas antibody in the p53 null cell line was associated with induction of the Fas/CD95 receptor by flow cytometry following treatment with these agents for 24 hours. Both 5-FU and Oxaliplatin were only able to significantly induce expression of the receptor in the HCT116 p53 wild-type cell line (FIG. 11A, B).

### EXAMPLE 11

### Fas/CD95 Cell Surface Expression in the p53 Mutant H630 and p53 Wild-Type RKO Cell Lines Following Treatment with the Chemotherapeutic Agents 5-FU, TDX, CPT-11 and Oxaliplatin

Induction of the Fas receptor in the p53 mutant H630 cell line was only seen with CPT-11. Neither 5-FU nor Oxaliplatin treatment for 48 hours caused significant upregulation of the receptor (FIG. 12A). In the p53 wild-type RKO cell line there was significant induction of the Fas receptor in response to IC50 doses of all three chemotherapeutic agents (FIG. 12A). When each of the cell lines was treated with these chemotherapy drugs for 24 hours followed by the anti-Fas antibody CH-11 for an additional 48 hours significant synergy was evident with the CPT-11 combination in the H630 cell line (FIG. 12B). When the RKO cell line was treated under similar conditions each of the three drugs displayed synergy with CH-11 (FIG. 12C).

### EXAMPLE 12

### The R175H and R248W p53 Mutant Cell Lines Show Similar Levels of Fas/CD95 Expression in Response to the Chemotherapeutic Agents 5-FU, TDX, CPT-11 and Oxaliplatin as the Isogenic HCT116 p53 Null Cell Line

As shown in FIG. 13, the R175H and R248W p53 mutant cell lines show similar levels of Fas/CD95 expression in response to the chemotherapeutic agents 5-FU, TDX, CPT-11 and Oxaliplatin as the isogenic HCT116 p53 null cell line. As shown in FIG. 14, synergistic cytotoxicity was demontsrated for combinations of a chemotherapeutic agent and CH-11, where the chemotherapeutic agent was 5-FU or CPT-11 but not oxaliplatin. The R175H and R248W p53 mutant cell lines show similar responses to the p53 null cell line for each drug combination tested.

The inventors have found that the Fas death receptor is highly up-regulated in response to 5-FU and the TS-targeted antifolates RTX and MTA in MCF-7 breast cancer and HCT116p53^{+/+} and RKO colon cancer cells. However, this was in itself not sufficient to activate caspase 8. To mimic the effects of immune effector cells in their in vitro model, the inventors used the agonistic Fas monoclonal antibody CH-11. The inventors found that CH-11 potently activated Fas-mediated cell death in 5-FU- and antifolate-treated cells. Furthermore, the interaction between CH-11 and each drug was highly synergistic. The inventors' results suggest that the Fas signalling pathway is an important mediator not only of 5-FU-induced cell death, but also of antifolate-induced cell death.

The inventors found that although FasL was not induced following drug treatment, it was highly expressed in MCF-7 cells. Many tumour cells overexpress FasL, and it has been postulated that tumour FasL induces apoptosis of Fas-sensitive immune effector cells, thereby inhibiting the antitumor immune response. This hypothesis has been supported by both in vitro and in vivo studies (24, 25). The strategy of overexpressing FasL requires that the tumour cells develop resistance to Fas-mediated apoptosis to prevent autocrine and paracrine induction of tumour cell death. Fas signalling may be inhibited by a Fas splice variant soluble Fas (sFas), which is a secreted protein that lacks the transmembrane domain of full-length Fas and may inhibit binding of FasL to Fas (26). Similarly, the Fas decoy receptor DcR3 is another secreted protein that binds to FasL with high affinity inhibiting its interaction with Fas (27). Downstream of Fas ligation, c-FLIP (FLICE-inhibitory protein) and FAP-1 (Fas-associated phosphatase-1) can inhibit caspase 8 recruitment and activation at the Fas DISC (28, 29). The lack of caspase 8 activation in response to treatment with 5-FU and the antifolates suggests that Fas-mediated apoptosis may be inhibited in MCF-7, HCT116 and RKO cancer cells. However, co-treatment with CH-11 was sufficient to overcome this resistance and activate Fas-mediated apoptosis.

The inventors' findings raise the possibility of using antimetabolite drugs in combination with anti-Fas antibodies as a novel anticancer strategy. Targeting Fas may be particularly useful against tumour cells that overexpress FasL and Fas pathway inhibitors, and which thereby evade Fas-mediated elimination by immune cells. However, systemic treatment with Fas antibodies or rFasL in mouse models has been shown to cause severe damage to liver and other organs (31). Some recent studies have focussed on local administration of rFasL, or the use of FasL-expressing vectors as gene therapy to overcome systemic toxicity (31). In addition, a novel agonistic Fas-targeted antibody HFE7A has been developed recently that was not hepatotoxic in murine models, suggesting that it may be possible to develop less toxic Fas-targeted antibodies (32).

Treatment with TS inhibitors has been shown to acutely induce TS expression in cell lines and tumours (18, 33). Furthermore, pre-clinical and clinical studies have found that TS is a key determinant of sensitivity to 5-FU, with high TS expression correlating with increased resistance (1, 34). The inventors therefore examined the effect of elevated TS expression on activation of Fas-mediated apoptosis in 5-FU- and antifolate-treated cells using a tetracycline-regulated TS expression system (M7TS90). Interestingly, the inventors found that activation of apoptosis by CH-11 in response to 5-FU was not affected by increased TS expression. In contrast, TS induction completely abrogated the synergistic interaction between both RTX and CH-11 and MTA and CH-11. These findings correlated with Fas expression, the up-regulation of which was almost completely abrogated by TS induction in RTX- and MTA-treated cells, but not 5-FU-treated cells. These results indicate that the primary locus of 5-FU cytotoxicity in this cell line was not TS inhibition. Indeed, the inventors' previous studies have suggested that misincorporation of fluoronucleotides into RNA was the primary cytotoxic effect of 5-FU in this line (6). Thus, despite expressing high levels of TS, certain tumours may still be sensitised to Fas-mediated apoptosis by 5-FU. However, high TS expression is likely to inhibit Fas-mediated apoptosis in response to folate-based TS inhibitors.

Several pre-clinical studies have demonstrated that loss of p53 function reduces cellular sensitivity to 5-FU (6, 21). Furthermore, a number of clinical studies have found that p53 mutations correlated with resistance to 5-FU, although other studies found no such association (34). The inventors assessed the effect of p53 inactivation on drug-induced Fas-mediated apoptosis in two p53 wild type and null isogenic cell line pairs: the MCF-7-derived M7TS90 and M7TS90-E6 lines, and the HCT116p53^{+/+} and HCT116p53^{-/-} lines. p53 inactivation attenuated Fas up-regulation in response to both drugs in both cell lines and inhibited the activation of apoptosis by CH-11 in 5-FU- and antifolate-treated cells, indicating that p53 is an important determinant of Fas-mediated apoptosis in response to these agents. Interestingly, some synergy was still observed between RTX and CH-11 in the HCT116p53^{-/-} cell line, although it was significantly reduced compared to the p53 wild type cell line. The inventors also examined activation of Fas-mediated apoptosis in response to the antimetabolites in the p53 mutant H630 colon cancer cell line. Similar to the HCT116p53^{-/-} cell line, little Fas induction was observed following drug treatment and no synergy was observed between 5-FU and CH-11. However, a statistically significant synergistic interaction was again observed between RTX and CH-11. The inventors' results surprisingly suggest that RTX (but not 5-FU) can sensitize at least some cancer cell lines with non-functional p53 to Fas-mediated apoptosis. Furthermore, this effect appears to be independent of Fas up-regulation, suggesting that factors other than increased Fas expression contribute to the sensitisation of tumour cells to Fas-mediated apoptosis in response to this agent.

In conclusion, the inventors have found that the agonistic Fas monoclonal antibody CH-11 dramatically increases the apoptotic response to 5-FU and TS-targeted antifolates in MCF-7, HCT116p53^{+/+} and RKO cells. Induction of exogenous TS abrogated this synergistic interaction for the antifolates but not 5-FU, however, the extent of the interaction was highly p53-dependent for each drug. The inventors' findings suggest that the Fas signalling pathway is an important regulator of 5-FU- and antifolate-mediated cell death and that targeting the Fas pathway in conjunction with either 5-FU or antifolates may have therapeutic potential.

Further, the inventors have surprisingly shown that, in contrast to other chemotherapies, antifolates such as TDX, topoisomerase-I inhibitors such as CPT-11 and, to a lesser extent, thymidylate synthase inhibitors such as 5-FU, provide a synergistic cytotoxic effect when used in combination therapies with death receptor ligands, such as CH-11, against cancers associated with a mutation in p53.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### REFERENCES

1. Longley, D. B., Harkin, D. P., and Johnston, P. G. 5-fluorouracil: mechanisms of action and clinical strategies. Nat Rev Cancer, 3: 330-338, 2003.
2. Hughes, L. R., Stephens, T. C., Boyle, F. T., and Jackman, A. L. Raltitrexed (Tomudex™), a highly polyglutamatable antifolate thymidylate synthase inhibitor, p. 147-165: Humana press, 1999.
3. Shih, C., Chen, V. J., Gossett, L. S., Gates, S. B., MacKellar, W. C., Habeck, L. L., Shackelford, K. A., Mendelsohn, L. G., Soose, D. J., Patel, V. F., Andis, S. L., Bewley, J. R., Rayl, E. A., Moroson, B. A., Beardsley, G. P., Kohler, W., Ratnam, M., and Schultz, R. M. LY231514, a pyrrolo[2,3-d]pyrimidine-based antifolate that inhibits multiple folate-requiring enzymes. Cancer Res, 57: 1116-1123, 1997.
4. Aherne, G. W., Hardcastle, A., Raynaud, F., and Jackman, A. L. Immunoreactive dUMP and TTP pools as an index of thymidylate synthase inhibition; effect of tomudex (ZD1694) and a nonpolyglutamated quinazoline antifolate (CB30900) in L1210 mouse leukaemia cells. Biochem Pharmacol, 51: 1293-1301, 1996.
5. Longley, D. B., Ferguson, P. R., Boyer, J., Latif, T., Lynch, M., Maxwell, P., Harkin, D. P., and Johnston, P. G. Characterization of a thymidylate synthase (TS)-inducible cell line: a model system for studying sensitivity to TS- and non-TS-targeted chemotherapies. Clin Cancer Res, 7: 3533-3539, 2001.
6. Longley, D. B., Boyer, J., Allen, W. L., Latif, T., Ferguson, P. R., Maxwell, P. J., McDermott, U., Lynch, M., Harkin, D. P., and Johnston, P. G. The role of thymidylate synthase induction in modulating p53-regulated gene expression in response to 5-fluorouracil and antifolates. Cancer Res, 62: 2644-2649, 2002.
7. Maxwell, P. J., Longley, D. B., Latif, T., Boyer, J., Allen, W., Lynch, M., McDermott, U., Harkin, D. P., Allegra, C. J., and Johnston, P. G. Identification of 5-fluorouracil-inducible target genes using cDNA microarray profiling. Cancer Research, 63, 2003.
12. Mosmann, T. Rapid calorimetric assay for cellular growth and survival: application to proliferation and cytotoxicity assays. J Immunol Methods, 65: 55-63, 1983.
13. Kern, D. H., Morgan, C. R., and Hildebrand-Zanki, S. U. In vitro pharmacodynamics of 1-beta-D-arabinofuranosylcytosine: synergy of antitumor activity with cis-diamminedichloroplatinum(II). Cancer Res, 48: 117-121, 1988.
14. Romanelli, S., Perego, P., Pratesi, G., Carenini, N., Tortoreto, M., and Zunino, F. In vitro and in vivo interaction between cisplatin and topotecan in ovarian carcinoma systems. Cancer Chemother Pharmacol, 41: 385-390, 1998.
15. Chou, T. C. and Talalay, P. Quantitative analysis of dose-effect relationships: the combined effects of multiple drugs or enzyme inhibitors. Adv Enzyme Regul, 22: 27-55, 1984.
16. Kano, Y., Ohnuma, T., Okano, T., and Holland, J. F. Effects of vincristine in combination with methotrexate and other antitumor agents in human acute lymphoblastic leukemia cells in culture. Cancer Res, 48: 351-356, 1988.
17. Salvesen, G. S. and Dixit, V. M. Caspase activation: the induced-proximity model. Proc Natl Acad Sci USA, 96: 10964-10967, 1999.
18. Chu, E., Koeller, D. M., Johnston, P. G., Zinn, S., and Allegra, C. J. Regulation of thymidylate synthase in human colon cancer cells treated with 5-fluorouracil and interferon-gamma. Mol Pharmacol, 43: 527-533, 1993.
19. Muller, M., Wilder, S., Bannasch, D., Israeli, D., Lehlbach, K., Li-Weber, M., Friedman, S. L., Galle, P. R., Stremmel, W., Oren, M., and Krammer, P. H. p53 activates the CD95 (APO-1/Fas) gene in response to DNA damage by anticancer drugs. J Exp Med, 188: 2033-2045, 1998.
20. Bennett, M., Macdonald, K., Chan, S. W., Luzio, J. P., Simari, R., and Weissberg, P. Cell surface trafficking of Fas: a rapid mechanism of p53-mediated apoptosis. Science, 282: 290-293, 1998.
21. Bunz, F., Hwang, P. M., Torrance, C., Waldman, T., Zhang, Y., Dillehay, L., Williams, J., Lengauer, C., Kinzler, K. W., and Vogelstein, B. Disruption of p53 in human cancer cells alters the responses to therapeutic agents. J Clin Invest, 104: 263-269, 1999.
24. O'Connell, J., Bennett, M. W., O'Sullivan, G. C., Collins, J. K., and Shanahan, F. The Fas counterattack: a molecular mechanism of tumor immune privilege. Mol Med, 3: 294-300, 1997.
25. Bennett, M. W., O'Connell, J., O'Sullivan, G. C., Brady, C., Roche, D., Collins, J. K., and Shanahan, F. The Fas counterattack in vivo: apoptotic depletion of tumor-infiltrating lymphocytes associated with Fas ligand expression by human esophageal carcinoma. J Immunol, 160: 5669-5675, 1998.
26. Ruberti, G., Cascino, I., Papoff, G., and Eramo, A. Fas splicing variants and their effect on apoptosis. Adv Exp Med Biol, 406: 125-134, 1996.
27. Pitti, R. M., Marsters, S. A., Lawrence, D. A., Roy, M., Kischkel, F. C., Dowd, P., Huang, A., Donahue, C. J., Sherwood, S. W., Baldwin, D. T., Godowski, P. J., Wood, W. I., Gurney, A. L., Hillan, K. J., Cohen, R. L., Goddard, A. D., Botstein, D., and Ashkenazi, A. Genomic amplification of a decoy receptor for Fas ligand in lung and colon cancer. Nature, 396: 699-703, 1998.
28. Krueger, A., Baumann, S., Krammer, P. H., and Kirchhoff, S. FLICE-inhibitory proteins: regulators of death receptor-mediated apoptosis. Mol Cell Biol, 21: 8247-8254, 2001.
29. Sato, T., Irie, S., Kitada, S., and Reed, J. C. FAP-1: a protein tyrosine phosphatase that associates with Fas. Science, 268: 411-415, 1995.
30. Jiang, S., Song, M. J., Shin, E. C., Lee, M. O., Kim, S. J., and Park, J. H. Apoptosis in human hepatoma cell lines by chemotherapeutic drugs via Fas-dependent and Fas-independent pathways. Hepatology, 29: 101-110, 1999.
31. Timmer, T., de Vries, E. G., and de Jong, S. Fas receptor-mediated apoptosis: a clinical application? J Pathol, 196: 125-134, 2002.
32. Ichikawa, K., Yoshida-Kato, H., Ohtsuki, M., Ohsumi, J., Yamaguchi, J., Takahashi, S., Tani, Y., Watanabe, M., Shiraishi, A., Nishioka, K., Yonehara, S., and Serizawa, N. A novel murine anti-human Fas mAb which mitigates lymphadenopathy without hepatotoxicity. Int Immunol, 12: 555-562, 2000.
33. Swain, S. M., Lippman, M. E., Egan, E. F., Drake, J. C., Steinberg, S. M., and Allegra, C. J. Fluorouracil and high-dose leucovorin in previously treated patients with metastatic breast cancer. J Clin Oncol, 7: 890-899, 1989.
34. Longley, D. B., McDermott, U., and Johnston, P. G. Clinical significance of prognostic and predictive markers in colorectal cancer. Pharmacogenomics J, 2: 209-216, 2002.
35. Munsch, D., Watanabe-Fukunaga, R., Bourdon, J. C., Nagata, S., May, E., Yonish-Rouach, E., and Reisdorf, P. Human and mouse Fas (APO-1/CD95) death receptor genes each contain a p53-responsive element that is activated by p53 mutants unable to induce apoptosis. J Biol Chem, 275: 3867-3872, 2000.
36. Oehm, A., et al., Purification and molecular cloning of the APO-1 cell surface antigen, a member of the tumor necrosis factor/nerve growth factor receptor superfamily. Sequence identity with the Fas antigen. J Biol Chem, 1992. 267(15): p. 10709-15.
37. Fischer, U., R. U. Janicke, and K. Schulze-Osthoff, Many cuts to ruin: a comprehensive update of caspase substrates. Cell Death Differ, 2003. 10(1): p. 76-100.
38. Hill, L. L., et al., Fas ligand: a sensor for DNA damage critical in skin cancer etiology. Science, 1999. 285(5429): p. 898-900.
39. Santi, D. V., C. S. McHenry, and H. Sommer, Mechanism of interaction of thymidylate synthetase with 5-fluorodeoxyuridylate. Biochemistry, 1974. 13(3): p. 471-81.
40. Danenberg, P. V., Thymidylate synthetase—a target enzyme in cancer chemotherapy. Biochim Biophys Acta, 1977. 473(2): p. 73-92.
41. Kufe, D. W. and P. P. Major, 5-Fluorouracil incorporation into human breast carcinoma RNA correlates with cytotoxicity. J Biol Chem, 1981. 256(19): p. 9802-5.
42. Dancey, J. and E. A. Eisenhauer, Current perspectives on camptothecins in cancer treatment. Br J Cancer, 1996. 74(3): p. 327-38.
43. Voigt, W., et al., Topoisomerase-I inhibitor SN-38 can induce DNA damage and chromosomal aberrations independent from DNA synthesis. Anticancer Res, 1998. 18(5A): p. 3499-505.
44. Chaney, S. G., The chemistry and biology of platinum complexes with the 1,2-diaminocyclohexane carrier ligand. Int J Oncol, 1995. 6: p. 1291-1305.
45. Woynarowski, J. M., et al., Oxaliplatin-induced damage of cellular DNA. Mol Pharmacol, 2000. 58(5): p. 920-7.
46. Jackman, A. L., et al., ICI D1694, a quinazoline antifolate thymidylate synthase inhibitor that is a potent inhibitor of L1210 tumor cell growth in vitro and in vivo: a new agent for clinical study. Cancer Res, 1991. 51(20): p. 5579-86.
47. NIH consensus conference. Adjuvant therapy for patients with colon and rectal cancer. Jama, 1990. 264(11): p. 1444-50.
48. Tournigand, C., et al., FOLFIRI followed by FOLFOX6 or the reverse sequence in advanced colorectal cancer: a randomized GERCOR study. J Clin Oncol, 2004. 22(2): p. 229-37.
49. Kong, X. T., H. Gao, and E. J. Stanbridge, Mechanisms of differential activation of target gene promoters by p53 hinge domain mutants with impaired apoptotic function. J Biol Chem, 2001. 276(35): p. 32990-3000.
50. Gurova, K. V., et al., Cooperation of two mutant p53 alleles contributes to Fas resistance of prostate carcinoma cells. Cancer Res, 2003. 63(11): p. 2905-12.
51. He, M., et al., A mutant P53 can activate apoptosis through a mechanism distinct from those induced by wild type P53. FEBS Lett, 2002. 517(1-3): p. 151-4.
52. Pugacheva, E. N., et al., Novel gain of function activity of p53 mutants: activation of the duTPase gene expression leading to resistance to 5-fluorouracil. Oncogene, 2002. 21(30): p. 4595-600.
53. Forrester, K., et al., Effects of p53 mutants on wild-type p53-mediated transactivation are cell type dependent. Oncogene, 1995. 10(11): p. 2103-11.

1-8. (canceled)

9. A method of killing cancer cells having a p53 mutation, said method comprising the separate, sequential or simultaneous administration to said cells of a therapeutically effective amount of a) a specific binding member which binds to a cell death receptor or a nucleic acid encoding said binding member and (b) a chemotherapeutic agent, wherein said chemotherapeutic agent is a topoisomerase inhibitor or a thymidylate synthase inhibitor.

10. A method of treating cancer cells having a p53 mutation comprising the separate, sequential or simultaneous administration to a mammal in need thereof of a therapeutically effective amount of a) a specific binding member which binds to a cell death receptor or a nucleic acid encoding said binding member and (b) a chemotherapeutic agent, wherein said chemotherapeutic agent is a topoisomerase inhibitor or a thymidylate synthase inhibitor.

11. The method according to claim 9 wherein the cancer is one or more of colorectal, breast, ovarian, cervical, gastric, lung, liver, skin and myeloid cancer.

12. The method according to claim 9 wherein the binding member is an antibody or a fragment thereof.

13. The method according to claim 9 wherein the death receptor is FAS.

14. The method according to claim 9 wherein the binding member is the anti-FAS antibody CH11.

15. The method according to claim 9 wherein said chemotherapeutic agent is an antifolate thymidylate synthase inhibitor or a topoisomerase-I inhibitor.

16. The method according to claim 9, wherein said chemotherapeutic agent is TDX or irinotecan (CPT-11).

17. The method according to claim 16 wherein said specific binding member and chemotherapeutic agent are provided in concentrations sufficient to produce an RI of greater than 1.5.

18. A product comprising a) a specific binding member which binds to a cell death receptor or a nucleic acid encoding said binding member and (b) a chemotherapeutic agent as a combined preparation for the simultaneous, separate or sequential use in the treatment of cancer, wherein said chemotherapeutic agent is a topoisomerase inhibitor or a thymidylate synthase inhibitor, and wherein the cancer cells comprise a p53 mutation.

19. A pharmaceutical composition for the treatment of cancer characterised by the presence of a p53 mutation, wherein the composition comprises a) a specific binding member which binds to a cell death receptor or a nucleic acid encoding said binding member and (b) a chemotherapeutic agent, wherein said chemotherapeutic agent is a topoisomerase inhibitor or a thymidylate synthase inhibitor and (c) a pharmaceutically acceptable excipient, diluent or carrier.

20. The product according to claim 18 wherein the cancer is one or more of colorectal, breast, ovarian, cervical, gastric, lung, liver, skin and myeloid cancer.

21. The product according to claim 18 wherein the binding member is an antibody or a fragment thereof.

22. The product according to claim 18 wherein the death receptor is FAS.

23. The product according to claim 18 wherein the binding member is the anti-FAS antibody CH11.

24. The product according to claim 18 wherein said chemotherapeutic agent is an antifolate thymidylate synthase inhibitor or a topoisomerase-I inhibitor.

25. The product according to claim 18 wherein said chemotherapeutic agent is TDX or irinotecan (CPT-11).

26. The product according to claim 25 wherein said specific binding member and chemotherapeutic agent are provided in concentrations sufficient to produce an RI of greater than 1.5.

27. A kit for the treatment of a cancer characterised by the presence of a p53 mutation, said kit comprising a) a specific binding member which binds to a cell death receptor or a nucleic acid encoding said binding member and (b) a chemotherapeutic agent, wherein said chemotherapeutic agent is a topoisomerase inhibitor or a thymidylate synthase inhibitor and (c) instructions for the administration of (a) and (b) separately, sequentially or simultaneously.

28. The pharmaceutical composition according to claim 19 wherein the cancer is one or more of colorectal, breast, ovarian, cervical, gastric, lung, liver, skin and myeloid cancer.

29. The pharmaceutical composition according to claim 19 wherein the binding member is an antibody or a fragment thereof.

30. The pharmaceutical composition according to claim 19 wherein the death receptor is FAS.

31. The pharmaceutical composition according to claim 19 wherein the binding member is the anti-FAS antibody CH11.

32. The pharmaceutical composition according to claim 19 wherein said chemotherapeutic agent is an antifolate thymidylate synthase inhibitor or a topoisomerase-I inhibitor.

33. The pharmaceutical composition according to claim 19 wherein said chemotherapeutic agent is TDX or irinotecan (CPT-11).

34. The pharmaceutical composition according to claim 25 wherein said specific binding member and chemotherapeutic agent are provided in concentrations sufficient to produce an RI of greater than 1.5.
